SEQUENCE LISTING (137 sequences)

SEQ ID NO: 1 - protein, 10 aa, Synthetic construct
GVYDGREHTV

SEQ ID NO: 2 - protein, 9 aa, Synthetic construct
KVLEHVVRV

SEQ ID NO: 3 - protein, 7 aa, synthetic construct
NIATNDY

SEQ ID NO: 4 - protein, 5 aa, synthetic construct
SGHAT

SEQ ID NO: 5 - protein, 5 aa, synthetic construct
GYKTK

SEQ ID NO: 6 - protein, 6 aa, synthetic construct
FQNNGV

SEQ ID NO: 7 - protein, 15 aa, Synthetic construct
CLVGGFYTGGFKTIF

SEQ ID NO: 8 - protein, 14 aa, Synthetic construct
CASSIRDNDEKLFF

SEQ ID NO: 9 - protein, 114 aa, Synthetic construct
LAKTTQPISMDSYEGQEVNITCSHNNIATNDYITWYQQFPSQGPRFIIQGYKTKVTNEVASLFIPADRKSSTLSLPRVSLSDTAVYYCLVGGFYTGGFKTIFGAGTRLFVKANI

SEQ ID NO: 10 - protein, 131 aa, Synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGGFYTGGFKTIFGAGTRLFVKANI

SEQ ID NO: 11 - protein, 291 aa, Synthetic construct
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSIRDNDEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 310 aa, Synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSIRDNDEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 13 - protein, 6 aa, Synthetic construct
YGGTVN

SEQ ID NO: 14 - protein, 5 aa, Synthetic construct
SNHLY

SEQ ID NO: 15 - protein, 8 aa, Synthetic construct
YFSGDPLV

SEQ ID NO: 16 - protein, 6 aa, Synthetic construct
FYNNEI

SEQ ID NO: 17 - protein, 13 aa, Synthetic construct
CAVGSGNTGKLIF

SEQ ID NO: 18 - protein, 12 aa, Synthetic construct
CAYDRDGYEQYF

SEQ ID NO: 19 - protein, 115 aa, Synthetic construct
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAVGSGNTGKLIFGQGTTLQVKPDI

SEQ ID NO: 20 - protein, 134 aa, Synthetic construct
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVGSGNTGKLIFGQGTTLQVKPDI

SEQ ID NO: 21 - protein, 291 aa, Synthetic construct
EPEVTQTPSHQVTQMGQEVILRCVPISNHLYFYWYRQILGQKVEFLVSFYNNEISEKSEIFDDQFSVERPDGSNFTLKIRSTKLEDSAMYFCAYDRDGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 22 - protein, 310 aa, Synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCAYDRDGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 6 aa, Synthetic construct
NSASDY

SEQ ID NO: 24 - protein, 5 aa, Synthetic construct
SGHDT

SEQ ID NO: 25 - protein, 7 aa, Synthetic construct
IRSNMDK

SEQ ID NO: 26 - protein, 6 aa, Synthetic construct
YYEEEE

SEQ ID NO: 27 - protein, 17 aa, Synthetic construct
CAEASPRQDTGRRALTF

SEQ ID NO: 28 - protein, 14 aa, Synthetic construct
CASSLWTGSGELFF

SEQ ID NO: 29 - protein, 118 aa, Synthetic construct
GESVGLHLPTLSVQEGDNSIINCAYSNSASDYFIWYKQESGKGPQFIIDIRSNMDKRQGQRVTVLLNKTVKHLSLQIAATQPGDSAVYFCAEASPRQDTGRRALTFGSGTRLQVQPNI

SEQ ID NO: 30 - protein, 138 aa, Synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEASPRQDTGRRALTFGSGTRLQVQPNI

SEQ ID NO: 31 - protein, 292 aa, Synthetic construct
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLWTGSGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 32 - protein, 311 aa, Synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLWTGSGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 33 - protein, 7 aa, Synthetic construct
NIATNDY

SEQ ID NO: 34 - protein, 5 aa, Synthetic construct
SGHAT

SEQ ID NO: 35 - protein, 5 aa, Synthetic construct
GYKTK

SEQ ID NO: 36 - protein, 6 aa, Synthetic construct
FQNNGV

SEQ ID NO: 37 - protein, 11 aa, Synthetic construct
CLVGGDEDMRF

SEQ ID NO: 38 - protein, 15 aa, Synthetic construct
CASSLEYGPTYEQYF

SEQ ID NO: 39 - protein, 110 aa, Synthetic construct
LAKTTQPISMDSYEGQEVNITCSHNNIATNDYITWYQQFPSQGPRFIIQGYKTKVTNEVASLFIPADRKSSTLSLPRVSLSDTAVYYCLVGGDEDMRFGAGTRLTVKPNI

SEQ ID NO: 40 - protein, 127 aa, Synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGGDEDMRFGAGTRLTVKPNI

SEQ ID NO: 41 - protein, 294 aa, Synthetic construct
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLEYGPTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 42 - protein, 313 aa, Synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLEYGPTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 43 - protein, 6 aa, Synthetic construct
ATGYPS

SEQ ID NO: 44 - protein, 5 aa, Synthetic construct
SGHVS

SEQ ID NO: 45 - protein, 7 aa, Synthetic construct
ATKADDK

SEQ ID NO: 46 - protein, 6 aa, Synthetic construct
FNYEAQ

SEQ ID NO: 47 - protein, 12 aa, Synthetic construct
CALSVDGQKLLF

SEQ ID NO: 48 - protein, 12 aa, Synthetic construct
CASSLADTEAFF

SEQ ID NO: 49 - protein, 113 aa, Synthetic construct
GDSVTQMEGPVTLSEEAFLTINCTYTATGYPSLFWYVQYPGEGLQLLLKATKADDKGSNKGFEATYRKETTSFHLEKGSVQVSDSAVYFCALSVDGQKLLFARGTMLKVDLNI

SEQ ID NO: 50 - protein, 132 aa, Synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSVDGQKLLFARGTMLKVDLNI

SEQ ID NO: 51 - protein, 289 aa, Synthetic construct
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLADTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 52 - protein, 308 aa, Synthetic construct
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLADTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 53 - protein, 7 aa, Synthetic construct
TSENNYY

SEQ ID NO: 54 - protein, 5 aa, Synthetic construct
MDHEN

SEQ ID NO: 55 - protein, 8 aa, Synthetic construct
QEAYKQQN

SEQ ID NO: 56 - protein, 6 aa, Synthetic construct
SYDVKM

SEQ ID NO: 57 - protein, 12 aa, Synthetic construct
CAFVDSGYALNF

SEQ ID NO: 58 - protein, 13 aa, Synthetic construct
CASSLDGARTQYF

SEQ ID NO: 59 - protein, 115 aa, Synthetic construct
AQTVTQSQPEMSVQEAETVTLSCTYDTSENNYYLFWYKQPPSRQMILVIRQEAYKQQNATENRFSVNFQKAAKSFSLKISDSQLGDTAMYFCAFVDSGYALNFGKGTSLLVTPHI

SEQ ID NO: 60 - protein, 135 aa, Synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFVDSGYALNFGKGTSLLVTPHI

SEQ ID NO: 61 - protein, 291 aa, Synthetic construct
DVKVTQSSRYLVKRTGEKVFLECVQDMDHENMFWYRQDPGLGLRLIYFSYDVKMKEKGDIPEGYSVSREKKERFSLILESASTNQTSMYLCASSLDGARTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 62 - protein, 310 aa, Synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLDGARTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 63 - protein, 7 aa, Synthetic construct
TSENNYY

SEQ ID NO: 64 - protein, 5 aa, Synthetic construct
MNHNY

SEQ ID NO: 65 - protein, 8 aa, Synthetic construct
QEAYKQQN

SEQ ID NO: 66 - protein, 6 aa, Synthetic construct
SVGAGI

SEQ ID NO: 67 - protein, 13 aa, Synthetic construct
CALLDSGNTPLVF

SEQ ID NO: 68 - protein, 12 aa, Synthetic construct
CASSYTNNEQFF

SEQ ID NO: 69 - protein, 255 aa, Synthetic construct
AQTVTQSQPEMSVQEAETVTLSCTYDTSENNYYLFWYKQPPSRQMILVIRQEAYKQQNATENRFSVNFQKAAKSFSLKISDSQLGDTAMYFCALLDSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 70 - protein, 275 aa, Synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCALLDSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 71 - protein, 290 aa, Synthetic construct
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYTNNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 72 - protein, 309 aa, Synthetic construct
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYTNNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - protein, 6 aa, Synthetic construct
DSAIYN

SEQ ID NO: 74 - protein, 5 aa, Synthetic construct
SNHLY

SEQ ID NO: 75 - protein, 7 aa, Synthetic construct
IQSSQRE

SEQ ID NO: 76 - protein, 6 aa, Synthetic construct
FYNNEI

SEQ ID NO: 77 - protein, 11 aa, Synthetic construct
CAVDAHARLMF

SEQ ID NO: 78 - protein, 10 aa, Synthetic construct
CASISGEQYF

SEQ ID NO: 79 - protein, 112 aa, Synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVDAHARLMFGDGTQLVVKPNI

SEQ ID NO: 80 - protein, 131 aa, Synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVDAHARLMFGDGTQLVVKPNI

SEQ ID NO: 81 - protein, 289 aa, Synthetic construct
EPEVTQTPSHQVTQMGQEVILRCVPISNHLYFYWYRQILGQKVEFLVSFYNNEISEKSEIFDDQFSVERPDGSNFTLKIRSTKLEDSAMYFCASISGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 82 - protein, 308 aa, Synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASISGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 83 - protein, 10 aa, Synthetic construct
GLYDGPVHEV

SEQ ID NO: 84 - protein, 10 aa, Synthetic construct
GLYDGPVCEV

SEQ ID NO: 85 - protein, 11 aa, Synthetic construct
FFVYDEPGHAV

SEQ ID NO: 86 - protein, 10 aa, Synthetic construct
GVYGGSVHEA

SEQ ID NO: 87 - protein, 10 aa, Synthetic construct
GVIDGHIYAV

SEQ ID NO: 88 - protein, 10 aa, Synthetic construct
FLSDPQVHTV

SEQ ID NO: 89 - protein, 10 aa, Synthetic construct
YTYDEAIHSV

SEQ ID NO: 90 - protein, 10 aa, Synthetic construct
FLLDGFPRTV

SEQ ID NO: 91 - protein, 10 aa, Synthetic construct
TVYGGYLCSV

SEQ ID NO: 92 - protein, 10 aa, Synthetic construct
VLFTGREFFV

SEQ ID NO: 93 - protein, 10 aa, Synthetic construct
DSGDGVTHTV

SEQ ID NO: 94 - protein, 10 aa, Synthetic construct
SQYSGQLHEV

SEQ ID NO: 95 - protein, 10 aa, Synthetic construct
LLKEGEEPTV

SEQ ID NO: 96 - protein, 10 aa, Synthetic construct
GLMDGSPHFL

SEQ ID NO: 97 - protein, 10 aa, Synthetic construct
VLEDGPWKTV

SEQ ID NO: 98 - protein, 10 aa, Synthetic construct
ALLDGRLQVV

SEQ ID NO: 99 - protein, 10 aa, Synthetic construct
GLFDGVPTTA

SEQ ID NO: 100 - protein, 10 aa, Synthetic construct
IIADGGIQTV

SEQ ID NO: 101 - protein, 9 aa, Synthetic construct
FLYTGEGTV

SEQ ID NO: 102 - protein, 10 aa, Synthetic construct
NQWDGTQHGV

SEQ ID NO: 103 - protein, 9 aa, Synthetic construct
KVLEILHRV

SEQ ID NO: 104 - protein, 9 aa, Synthetic construct
KVLERVNAV

SEQ ID NO: 105 - protein, 9 aa, Synthetic construct
KILEDVVGV

SEQ ID NO: 106 - protein, 9 aa, Synthetic construct
KVLETLVTV

SEQ ID NO: 107 - protein, 9 aa, Synthetic construct
FLLETVVRV

SEQ ID NO: 108 - protein, 9 aa, Synthetic construct
KVLGIVVGV

SEQ ID NO: 109 - protein, 9 aa, Synthetic construct
KILEALQRV

SEQ ID NO: 110 - protein, 9 aa, Synthetic construct
KLLEQVNRI

SEQ ID NO: 111 - protein, 9 aa, Synthetic construct
KVLDKVFRA

SEQ ID NO: 112 - protein, 9 aa, Synthetic construct
KLLDLQVRV

SEQ ID NO: 113 - protein, 9 aa, Synthetic construct
IVMEHVVFL

SEQ ID NO: 114 - protein, 9 aa, Synthetic construct
ILDEHVQRV

SEQ ID NO: 115 - protein, 9 aa, Synthetic construct
KVTHAVVTV

SEQ ID NO: 116 - protein, 9 aa, Synthetic construct
KLLEKVRKV

SEQ ID NO: 117 - protein, 9 aa, Synthetic construct
VALEHFVLV

SEQ ID NO: 118 - protein, 9 aa, Synthetic construct
KVLNKVITV

SEQ ID NO: 119 - protein, 9 aa, Synthetic construct
KVQEQVHKV

SEQ ID NO: 120 - protein, 9 aa, Synthetic construct
KVWGNVVTV

SEQ ID NO: 121 - protein, 9 aa, Synthetic construct
SLLGHVIRL

SEQ ID NO: 122 - protein, 9 aa, Synthetic construct
ILLEHKVVL

SEQ ID NO: 123 - protein, 64 aa, Synthetic construct
KCMRVDTSEACMILNLTPGACVMIYFKWQEHCYFVDTSEPGACMILYFHKRWQNVIREVTSGAC

SEQ ID NO: 124 - protein, 59 aa, Synthetic construct
KVIRVTSCILYLSPGACVMIYFHRWQNEHTFVSGACMILYVCIFWRVTSEPGACMILFQ

SEQ ID NO: 125 - protein, 58 aa, Synthetic construct
GDTSAIYFNVIYDGMILYFHRWQNRPIKECRHPYWTDSEPGACVMILYFHKQNVCILF

SEQ ID NO: 126 - protein, 90 aa, Synthetic construct
GDTSEPACVMILYFHKRWQNVTSAMILYQYFWDMNGTSPACVMILYFHKRWQNRPIKEDCMLYFHQNHTSPGACVMILHWQNVTACMILF

SEQ ID NO: 127 - protein, 10 aa, Synthetic construct
GLYDGREHSV

SEQ ID NO: 128 - protein, 10 aa, Synthetic construct
GVYAGREHFV

SEQ ID NO: 129 - protein, 10 aa, Synthetic construct
EVYDGREHSA

SEQ ID NO: 130 - protein, 10 aa, Synthetic construct
GVYAGREHFL

SEQ ID NO: 131 - protein, 10 aa, Synthetic construct
GLYDGMEHLI

SEQ ID NO: 132 - protein, 10 aa, Synthetic construct
VVDDGVEHTI

SEQ ID NO: 133 - protein, 10 aa, Synthetic construct
YLAWIREHTV

SEQ ID NO: 134 - protein, 10 aa, Synthetic construct
MIEDGKIHTV

SEQ ID NO: 135 - protein, 10 aa, Synthetic construct
LVYYLKEHTL

SEQ ID NO: 136 - protein, 10 aa, Synthetic construct
FVDDGTETHF

SEQ ID NO: 137 - protein, 10 aa, Synthetic construct
GEIDGNKVTL

(19) United States
(12) Patent Application Publication
(10) Pub. No.: US 2026/0085105 A1
(43) Pub. Date: Mar. 26, 2026

(54) MAGEA4 SPECIFIC T CELL RECEPTORS

(71) Applicant: AMGEN INC., Thousand Oaks, CA (US)

(72) Inventors: Sungeun KIM, San Mateo, CA (US); Matthew James RARDIN, San Bruno, CA (US)

(73) Assignee: AMGEN INC., Thousand Oaks, CA (US)

(21) Appl. No.: 18/876,904

(22) PCT Filed: Jun. 23, 2023
(86) PCT No.: PCT/US2023/026122

§ 371 Date: Dec. 19, 2024

Related U.S. Application Data
(60) Provisional application No. 63/355,559, filed on Jun. 24, 2022.

(51) Int. Cl. C07K 14/725; A61K 35/17; A61K 40/11; A61K 40/32; A61K 40/42; A61P 35/00; C07K 14/54; C12N 5/0783; C12N 15/867

(52) U.S. Cl. CPC C07K 14/7051; A61K 35/17; A61K 40/11; A61K 40/32; A61K 40/4268; A61P 35/00; C07K 14/5434; C12N 5/0636; C12N 15/867; C07K 2317/33; C07K 2317/565; C07K 2317/72; C12N 2740/15043

(57) ABSTRACT

Provided herein are recombinant T-cell receptors (TCRs) that can selectively recognize the MAGE-A4-derived peptide GVYDGEEHSV or KVEEHVVRV when presented by HLA-A*0201 sufficiently to activate the recombinant T cell. TCRs provided herein were thoroughly screened for lack of cross-reactivity with similar peptides that may be presented by normal cells or tissue and for alloreactivity.

### CROSS REFERENCE TO RELATED APPLICATIONS

This application is a national stage application of International Application No. PCT/US2023/026122, filed on Jun. 23, 2023, which claims the benefit under 35 U.S.C. § 119(e) of U.S. Provisional Application No. 63/355,559, filed on Jun. 24, 2022.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 123 KB XML file named “10175-WO01-SEC_Sequence_Listing. XML”; created on Oct. 12, 2023.

### BACKGROUND

Adoptive T cell therapies provide tremendous opportunities to treat cancer. Chimeric antigen receptor (CAR)-T cell therapy is an approved adoptive T cell therapy for hematological malignancy but has a limited range of targets due to its recognition to only cell surface antigens constituting ˜25% of the genome. Unlike CAR-T cells, TCR-T cells engineered to express the T cell receptors (TCR) specific to tumor antigens can exploit a broader range of targets for multiple cancer indications because TCR-T cells can recognize the peptide-MHC complexes (pMHC) derived from intracellular proteins constituting ˜75% of the genome. Intracellular proteins are processed and presented by major histocompatibility complex (MHC) as pMHC complexes.

Cancer-testis antigens (CTA) are attractive targets for cancer immunotherapy including TCR-T cell therapy due to their restricted expression in germ cells and aberrant reactivation in various cancers, and their immunogenic properties. Germ cells such as testis (immune-privileged sites) do not usually express HLA class I/II molecules, allowing them to evade attack from the immune system. MAGEA4 is a type I MAGE protein, a family of homologous proteins known to associate with E3 RING ligases to modulate protein ubiquitination. Consistent with this behavior, MAGEA4 has recently been described to contribute to oncogenesis by acting in concert with the E3 ligase RAD18 to promote ubiquitination of PCNA to facilitate trans-lesion synthesis, an error-prone method of DNA repair that may contribute to mutational load and oncogenesis in cancerous tissues.

While TCR-T cells are shown to be very potent and sensitive modality for tumor-specific peptide-MHC targets, a TCR can recognize multiple peptides. DNA rearrangement required for TCR formation generates a certain number of T cells that recognize self-antigens. During early T cell development, self-reactive T cells are negatively selected and eliminated in the medulla of the thymus through a promiscuous expression of a wide range of self-antigens in medullary thymic epithelial cells. This negative selection in the thymus functions as the major mechanism of central tolerance and shapes the T cell repertoire to avoid autoimmunity. TCRs that are engineered to increase their affinity for certain pMHC or to introduce cross-reactivity to multiple pMHC do not have the benefit of the negative selection that occurs in the thymus. It is noteworthy that affinity-enhanced MAGE-A3 TCR-T cells led to fatal toxicity due to cross-reactivity to Titin expressed in cardiac muscles (Cameron et al., Sci Transl Med. 2013 5(197)).

### SUMMARY

Identification of TCR sequences recognizing tumor-specific antigens has been shown to be very challenging in the field particularly due to rarity of tumor-specific T cells in patient blood, difficulty in expanding a very small number of tumor-specific T cell clones ex vivo, and potential exhaustion or suppression of tumor-specific T cells in tumor-infiltrating lymphocytes (TILs). Despite these challenges, provided herein are TCR sequences specific to two MAGEA4 peptide-MHC (KVLEHVVRV/HLA-A*02:01, GVYDGREHTV/HLA-A*02:01) identified through a healthy donor blood and an ex vivo stimulation method. As demonstrated in the Examples herein, the exemplary TCR-T cells recognizing the tumor-specific MAGEA4 pMHC can be highly potent therapeutics for the treatment of MAGEA4/HLA-A*02:01+ tumors by exerting cytotoxicity and producing cytokines. These TCR-T cell therapies will be a significant treatment option for a wide variety of cancer indications, for example, non-small cell lung cancers (NSCLC).

TCR-T cells are the most potent and sensitive modality in vitro for pMHC targets. The TCR-T cells provided herein display high potency against even very low target-expressing cells. This high potency of TCR-T cells comes from the complex of the transduced TCR and endogenous CD3 subunits. In some embodiments, to enhance in vivo efficacy, exemplary TCR-T cells comprise an activation-dependent IL12 payload that is incorporated into a TCR-T construct. In some embodiments, the IL12 expression is regulated by TCR activation under a composite promoter containing six NFAT (nuclear factor of activated T cells) response elements linked to a minimal IL-2 promoter. In these embodiments, when TCR-T-IL12 cells encounter tumor antigens, IL12 is produced. This strategy has been shown to enhance the efficacy of adoptive T cell therapy in vivo and therefore could decrease potential clinical dose by 10-100×.

In a first aspect, the invention is an expression vector comprising a nucleic acid sequence encoding a T-cell receptor (TCR) alpha chain and a TCR beta chain, wherein the TCR alpha chain and TCR beta chain are selected from the group consisting of:
a. a TCR alpha chain comprising a CDR1, 2, and 3 sequence comprising an amino acid sequence set forth in SEQ ID NO:3, 5, and 7, respectively, and a TCR beta chain comprising a CDR1, 2, and 3 sequence comprising an amino acid sequence set forth in SEQ ID NO: 4, 6, and 8, respectively;
b. a TCR alpha chain comprising a CDR1, 2, and 3 sequence comprising an amino acid sequence set forth in SEQ ID NO:13, 15, and 17, respectively, and a TCR beta chain comprising a CDR1, 2, and 3 sequence comprising an amino acid sequence set forth in SEQ ID NO: 14, 16, and 18, respectively;
c. a TCR alpha chain comprising a CDR1, 2, and 3 sequence comprising an amino acid sequence set forth in SEQ ID NO:23, 25, and 27, respectively, and a TCR beta chain comprising a CDR1, 2, and 3 sequence comprising an amino acid sequence set forth in SEQ ID NO: 24, 26, and 28, respectively;
d. a TCR alpha chain comprising a CDR1, 2, and 3 sequence comprising an amino acid sequence set forth in SEQ ID NO:33, 35, and 37, respectively, and a TCR beta chain comprising a CDR1, 2, and 3 sequence comprising an amino acid sequence set forth in SEQ ID NO: 34, 36, and 38, respectively;
e. a TCR alpha chain comprising a CDR1, 2, and 3 sequence comprising an amino acid sequence set forth in SEQ ID NO:43, 45, and 47, respectively, and a TCR beta chain comprising a CDR1, 2, and 3 sequence comprising an amino acid sequence set forth in SEQ ID NO: 44, 46, and 48, respectively;
f. a TCR alpha chain comprising a CDR1, 2, and 3 sequence comprising an amino acid sequence set forth in SEQ ID NO:53, 55, and 57, respectively, and a TCR beta chain comprising a CDR1, 2, and 3 sequence comprising an amino acid sequence set forth in SEQ ID NO: 54, 56, and 58, respectively;
g. a TCR alpha chain comprising a CDR1, 2, and 3 sequence comprising an amino acid sequence set forth in SEQ ID NO:63, 65, and 67, respectively, and a TCR beta chain comprising a CDR1, 2, and 3 sequence comprising an amino acid sequence set forth in SEQ ID NO: 64, 66, and 68, respectively; and
h. a TCR alpha chain comprising a CDR1, 2, and 3 sequence comprising an amino acid sequence set forth in SEQ ID NO:73, 75, and 77, respectively, and a TCR beta chain comprising a CDR1, 2, and 3 sequence comprising an amino acid sequence set forth in SEQ ID NO: 74, 76, and 78, respectively.

In another aspect, the invention is an expression vector comprising a nucleic acid sequence encoding a T-cell receptor (TCR) alpha chain and a TCR beta chain, wherein the TCR alpha chain and TCR beta chain are selected from the group consisting of:
a. an amino acid sequence set forth in SEQ ID NO:9 or 10 and the TCR beta chain comprises an amino acid sequence set forth in SEQ ID NO:11 or 12;
b. an amino acid sequence set forth in SEQ ID NO: 19 or 20 and the TCR beta chain comprises an amino acid sequence set forth in SEQ ID NO:21 or 22;
c. an amino acid sequence set forth in SEQ ID NO:29 or 30 and the TCR beta chain comprises an amino acid sequence set forth in SEQ ID NO:31 or 32;
d. an amino acid sequence set forth in SEQ ID NO:39 or 40 and the TCR beta chain comprises an amino acid sequence set forth in SEQ ID NO:41 or 42;
e. an amino acid sequence set forth in SEQ ID NO:49 or 50 and the TCR beta chain comprises an amino acid sequence set forth in SEQ ID NO:51 or 52;
f. an amino acid sequence set forth in SEQ ID NO:59 or 60 and the TCR beta chain comprises an amino acid sequence set forth in SEQ ID NO:61 or 62;
g. an amino acid sequence set forth in SEQ ID NO:69 or 70 and the TCR beta chain comprises an amino acid sequence set forth in SEQ ID NO:71 or 72; and
h. an amino acid sequence set forth in SEQ ID NO: 79 or 80 and the TCR beta chain comprises an amino acid sequence set forth in SEQ ID NO:81 or 82.

In another aspect, the invention is a cell expressing a recombinant T-cell receptor (TCR), said TCR comprising a TCR alpha chain and a TCR beta chain, wherein the TCR alpha chain and TCR beta chain are selected from the group consisting of:
a. a TCR alpha chain comprising a CDR1, 2, and 3 sequence comprising an amino acid sequence set forth in SEQ ID NO:3, 5, and 7, respectively, and a TCR beta chain comprising a CDR1, 2, and 3 sequence comprising an amino acid sequence set forth in SEQ ID NO: 4, 6, and 8, respectively;
b. a TCR alpha chain comprising a CDR1, 2, and 3 sequence comprising an amino acid sequence set forth in SEQ ID NO:13, 15, and 17, respectively, and a TCR beta chain comprising a CDR1, 2, and 3 sequence comprising an amino acid sequence set forth in SEQ ID NO: 14, 16, and 18, respectively;
c. a TCR alpha chain comprising a CDR1, 2, and 3 sequence comprising an amino acid sequence set forth in SEQ ID NO:23, 25, and 27, respectively, and a TCR beta chain comprising a CDR1, 2, and 3 sequence comprising an amino acid sequence set forth in SEQ ID NO: 24, 26, and 28, respectively;
d. a TCR alpha chain comprising a CDR1, 2, and 3 sequence comprising an amino acid sequence set forth in SEQ ID NO:33, 35, and 37, respectively, and a TCR beta chain comprising a CDR1, 2, and 3 sequence comprising an amino acid sequence set forth in SEQ ID NO: 34, 36, and 38, respectively;
e. a TCR alpha chain comprising a CDR1, 2, and 3 sequence comprising an amino acid sequence set forth in SEQ ID NO:43, 45, and 47, respectively, and a TCR beta chain comprising a CDR1, 2, and 3 sequence comprising an amino acid sequence set forth in SEQ ID NO: 44, 46, and 48, respectively;
f. a TCR alpha chain comprising a CDR1, 2, and 3 sequence comprising an amino acid sequence set forth in SEQ ID NO:53, 55, and 57, respectively, and a TCR beta chain comprising a CDR1, 2, and 3 sequence comprising an amino acid sequence set forth in SEQ ID NO: 54, 56, and 58, respectively;
g. a TCR alpha chain comprising a CDR1, 2, and 3 sequence comprising an amino acid sequence set forth in SEQ ID NO:63, 65, and 67, respectively, and a TCR beta chain comprising a CDR1, 2, and 3 sequence comprising an amino acid sequence set forth in SEQ ID NO: 64, 66, and 68, respectively; and
h. a TCR alpha chain comprising a CDR1, 2, and 3 sequence comprising an amino acid sequence set forth in SEQ ID NO:73, 75, and 77, respectively, and a TCR beta chain comprising a CDR1, 2, and 3 sequence comprising an amino acid sequence set forth in SEQ ID NO: 74, 76, and 78, respectively.

In another aspect, the invention is a cell expressing a recombinant T-cell receptor (TCR), said TCR comprising a TCR alpha chain and a TCR beta chain, wherein the TCR alpha chain and TCR beta chain are selected from the group consisting of:
a. an amino acid sequence set forth in SEQ ID NO:9 or 10 and the TCR beta chain comprises an amino acid sequence set forth in SEQ ID NO:11 or 12;
b. an amino acid sequence set forth in SEQ ID NO: 19 or 20 and the TCR beta chain comprises an amino acid sequence set forth in SEQ ID NO:21 or 22;
c. an amino acid sequence set forth in SEQ ID NO:29 or 30 and the TCR beta chain comprises an amino acid sequence set forth in SEQ ID NO:31 or 32;
d. an amino acid sequence set forth in SEQ ID NO:39 or 40 and the TCR beta chain comprises an amino acid sequence set forth in SEQ ID NO:41 or 42;
e. an amino acid sequence set forth in SEQ ID NO:49 or 50 and the TCR beta chain comprises an amino acid sequence set forth in SEQ ID NO:51 or 52;
f. an amino acid sequence set forth in SEQ ID NO:59 or 60 and the TCR beta chain comprises an amino acid sequence set forth in SEQ ID NO:61 or 62;
g. an amino acid sequence set forth in SEQ ID NO:69 or 70 and the TCR beta chain comprises an amino acid sequence set forth in SEQ ID NO:71 or 72; and
h. an amino acid sequence set forth in SEQ ID NO:79 or 80 and the TCR beta chain comprises an amino acid sequence set forth in SEQ ID NO:81 or 82.

### BRIEF DESCRIPTION OF THE DRAWINGS

FIG. 1. MAGEA4 is a tumor specific antigen that is broadly expressed in a broad range of solid tumors. (A) TCGA and internal RNA-seq data for MAGEA4 mRNA expression in a variety of cancers. (B) Body map RNA-seq data for MAGEA4 mRNA expression in human normal tissues. MAGEA4 expression is extremely restricted in normal tissues to the male reproductive system. (C) MAGE-A4 immunohistochemistry (IHC) by OTI1F9 monoclonal Ab shows that within a tumor of NSCLC-squamous, MAGE-A4 protein is expressed in the majority of tumor cells. The representative IHC stains of NSCLC-squamous tumors show 100% MAGE-A4 positive tumor cells and 3+ intense staining.

FIG. 2. RNA-seq and mass spectrometry (MS) of NSCLC specimens quantifying detectable HLA-A*02:01 bound MAGEA4 target peptides. High levels of MAGE-A4 FPKM mRNA expression are generally associated with detectable MAGEA4 target peptide presentation.

FIG. 3. Estimation of annual patient population in specified cancer indications. Annually treatable patient population was estimated based on pMHC target frequency×new cases per year in U.S. populations. The pMHC target frequency in each cancer indication was calculated by MAGE-A4 mRNA expression frequency×HLA-A*02:01 carrier frequency in U.S. populations (0.41). The MAGE-A4 mRNA levels (>1 FPKM) in various solid tumors were derived from TCGA data.

FIG. 4. Workflow for identifying MAGE-A4 pMHC-specific TCRs from rare T cell clones of healthy HLA-A*02:01+ donor PBMCs. (A) MAGE-A4 pMHC-specific T cells were stimulated and expanded via co-culture with MAGE-A4 peptide pulsed autologous APCs. MAGE-A4 pMHC-specific T cells were sorted for scRNAseq to identify MAGE-A4 pMHC-specific TCR sequences and functional validation by IFNγ ELISPOT. (B) Representative screen results demonstrate that a positive donor A showed the enriched MAGE-A4 pMHC-specific T cells after multiple ex vivo stimulation, whereas a negative donor B did not have Dex+ T cells. (C) MAGE-A4 pMHC-specific T cells are sorted and validated for antigen specificity by IFNγ ELISPOT.

FIG. 5. MAGE-A4 TCR activity measurement through a Jurkat activation assay. (A) T2 cells were loaded with target MAGEA4 peptide and co-cultured with TCR/GFP transfected Jurkat cells. TCR potency was evaluated by quantifying the CD69 upregulation on Jurkat cells for KVLEHVVRV pMHC TCRs. (B) Summary of measured TCR potency determined by the T2 peptide titration assay.

FIG. 6. Transduction of TCR-IL12 constructs into primary human T cells. (A) The TCR-T-IL12 lentiviral construct contains TCRα and TCRβ chains with a linker of furin cleavage site-SGSG-T2A under EF1α promoter, and IL12 payload under a composite promoter containing six NFAT (nuclear factor of activated T cells) response elements linked to a minimal IL-2 promoter. (B) Summary of transduction efficiencies for top eight TCRs as determined through flow cytometric analysis. T cell subset frequencies (%) are presented as an average of transduction efficiencies from TCR-Ts generated from two human donors.

FIG. 7. MAGEA4 TCR/IL-12 T cell cytotoxic activity against peptide loaded T2 cells. TCR-Ts using primary human T cells were tested using a T2 peptide titration assay. Representative T cell dependent cellular cytotoxicity (TDCC) assay, T2/peptide loading assay were shown for GVYDGREHTV pMHC (A) and KVLEHVVRVV pMHC (B) TCR-Ts. T cells were ranked by cytotoxic potency to identify the top 8 candidate TCRs (C).

FIG. 8. Top 20 similar peptides for GVYDGREHTV and KVLEHVVRV were identified and used to evaluate TCR cross-reactivity. T2 cells were pre-incubated with 10⁻⁵M of relevant peptide and co-cultured with corresponding top 8 TCR-Ts for 48 hours. Representative of TCR-Ts generated from 2 different donors.

FIG. 9. Sequence identity between target MAGEA4 peptides (GVY and KVL) and homologous MAGEA8 peptide. KVLEHVVRV peptide sequence is 100% identical in MAGEA4 and MAGEA8.

FIG. 10. Cross-reactivity of GVYDGREHTV-MHC specific TCRs against MAGEA8 peptide. (A) T2 cells were loaded with MAGEA8 peptide GLYDGREHSV at indicated concentrations and incubated with TCR-Ts for 48 h before evaluation of TDCC. (B) Summary of TCR potency data. Greater than ˜1000-fold difference in EC50 between the MAGEA4 and MAGEA8 peptides was observed for top four TCRs. Representative of experiments with TCR-Ts generated from two donors (8316 ad 12665).

FIG. 11. TDCC activity of top TCR-Ts against MAGEA4+HLA-A*02:01+ cancer cell lines. The top 8 TCR-Ts identified by T2 peptide titration potency assays were further evaluated in cancer cell killing assays. Highly potent cytolytic activities close to 100% specific killing were observed for MAGEA4+HLA-A*02:01+ cancer cell lines U266B1 (MAGEA4 FPKM 213.85) (A) and SCaBER (MAGEA4 FPKM 172) (B). Evaluated potency metrics are summarized and presented. Representative of experiments were shown using TCR-Ts generated from two donors (C and D). MAGEA4 expression in each cell line is derived from the Cancer Cell Line Encyclopedia (CCLE) and presented as FPKM.

FIG. 12. Top five identified TCRs were evaluated based on their potency in cancer cell line killing assays (A). The expression levels of MAGEA4 and HLA-A in cancer cell lines are derived from the CCLE and presented in FPKM. In some cell lines, HLA-A*02:01 bound MAGEA4 peptide KVLEHVVRV was quantified by mass spectrometry and is presented as copies per cell. Top TCR-Ts demonstrated cytolytic activity against a large set of MAGEA4+HLA-A*02:01 cell lines but did not kill the MAGEA4-HLA-A*02:01+ cell line CFPAC1. Potency statistics are summarized and presented in (B). Representative of experiments performed with TCR-Ts from three donors.

FIG. 13. Potency assays of off target peptides identified by the similar peptide screen. Putative cross-reactive peptides for TCR23 and TCR24 were evaluated in a TDCC/T2 peptide titration assay (A-B). Viability of T2 cells loaded with MAGEA4 target GVY peptide (GVY) is presented as a positive control. Peptides with a potency gap cutoff of <10³ fold in EC50 over the target peptide were considered for further risk assessment. No putative cross-reactivity risks were found in KVL reactive TCRs. Representative of experiments performed with TCR-Ts from three donors.

FIG. 14. Evaluation of TCR reactivity against human normal cells. (A) Top 4 TCR-Ts (circles) or RFP+IL12 T cell controls (squares) were co-cultured with a panel of human normal primary cells or iPSC-derived cell lines (MAGEA4-HLA-A*02:01+) representative of vital organs, including bronchial epithelial cells (hBEpC), tracheal epithelial cells (hTEpC), dermal microvascular endothelial cells (HDMEC), keratinocytes, hepatocytes, renal proximal tubule epithelial cells (RPTEC), iPSC-derived astrocytes, cardiomyocytes, and GABA neurons. Caspase 3/7 activity was measured over time using the Incucyte system. (B, C) Minimal or negligible reactivity over baseline was observed for TCR2 and TCR23 in all cases, while TCR10 and TCR24 exhibit clear cytotoxic activity against multiple normal cells.

FIG. 15. Summary of alloreactivity assessment. TCR-T-IL12 cells were co-cultured with each of 34 BLCLs representing highly frequent MHC Class I alleles. The HLA-A*02:01⁺ U266B1 cell line pulsed with the relevant MAGE-A4 peptide (KVL: KVLEHVVRV or GVY: GVYGDREHTV) served as a positive control for each TCR-T-IL12 cell. Secreted IFNγ (A), granzyme B (B), TNFα (C), and IL-12p70 (D) were evaluated as measures of potential alloreactivity by comparison to levels in response to co-culture with IL12-RFP control T cells. For mock transduced T cells, cytokine and granzyme B changes are shown in comparison to IL12-RFP cells at an effector:target ratio of 10:1.

FIG. 16. Cross-reactivity with additional HLA-A*02 alleles can broaden patient population. Top TCR-Ts recognize target peptide presented on additional HLA-A alleles with high homology to HLA-A*02:01. Homologous HLA-A alleles were overexpressed on HLA-A⁻ CIR cells, which were subsequently loaded with target KVL or GVY MAGEA4 peptide for use in TDCC assays. TCR2 and TCR23 both demonstrated cytolytic activity against the target peptide loaded CIR cells expressing HLA-A*02:05 and HLA-A*02:07, suggestive of potential inclusion of HLA-A*02:05 and HLA-A*02:07 patients for these TCR-T cell therapies.

### DETAILED DESCRIPTION

The section headings used herein are for organizational purposes only and are not to be construed as limiting the subject matter described. All references cited within the body of this specification are expressly incorporated by reference in their entirety.

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manual, 3rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

T cell receptors (TCRs) are naturally expressed by CD4+ and CD8+ T cells. TCRs are designed to recognize short peptide antigens that are displayed on the surface of antigen presenting cells in complex with Major Histocompatibility Complex (MHC) molecules (in humans, MHC molecules are also known as Human Leukocyte Antigens, or HLA) (Davis, et al., (1998), Annu Rev Immunol 16: 523-544.). CD8+ T cells, which are also termed cytotoxic T cells, specifically recognize peptides bound to MHC class I and are generally responsible for finding and mediating the destruction of infected or cancerous cells.

Therapeutic TCRs may be used, for example, as soluble targeting agents for the purpose of delivering cytotoxic or immune effector agents to the tumor (Lissin, et al., (2013). “High-Affinity Monocloncal T-cell receptor (mTCR) Fusions. Fusion Protein Technologies for Biopharmaceuticals: Applications and Challenges”. S. R. Schmidt, Wiley; Boulter, et al., (2003), Protein Eng 16(9): 707-711; Liddy, et al., (2012), Nat Med 8: 980-987), or alternatively they may be used to engineer T cells for adoptive therapy (June, et al., (2014), Cancer Immunol Immunother 63(9): 969-975). It is desirable that TCRs for immunotherapeutic use are able to strongly recognize the target antigen, by which it is meant that the TCR should possess a high affinity and/or long binding half-life for the target antigen in order to exert a potent response. TCRs as they exist in nature typically have low affinity for target antigen (low micromolar range), thus it is often necessary to identify mutations, including but not limited to substitutions, insertions and/or deletions, that can be made to a given TCR sequence in order to improve antigen binding. For use as soluble targeting agents TCR antigen binding affinities in the nanomolar to picomolar range and with binding half-lives of several hours are preferable. It is also desirable that therapeutic TCRs demonstrate a high level of specificity for the target antigen to mitigate the risk of toxicity in clinical applications resulting from off-target binding. Such high specificity may be especially challenging to obtain given the natural degeneracy of TCR antigen recognition (Wooldridge, et al., (2012), J Biol Chem 287(2): 1168-1177; Wilson, et al., (2004), Mol Immunol 40(14-15): 1047-1055). Finally, it is desirable that therapeutic TCRs are able to be expressed and purified in a highly stable form.

The variable domain of each chain is located N-terminally and comprises three Complementarity Determining Regions (CDRs) embedded in a framework sequence. The CDRs comprise the recognition site for peptide-MHC binding. There are several genes coding for alpha chain variable (Va) regions and several genes coding for beta chain variable (Vβ) regions. These genes are distinguished by their framework, CDR1 and CDR2 sequences, and by a partly defined CDR3 sequence. The Va and ν genes are referred to in IMGT nomenclature by the prefixes TRAV and TRBV respectively (Folch and Lefranc, (2000), Exp Clin Immunogenet 17(1): 42-54; Scaviner and Lefranc, (2000), Exp Clin Immunogenet 17(2): 83-96; LeFranc and LeFranc, (2001), “T cell Receptor Factsbook”, Academic Press). Likewise there are several joining or J genes, termed TRAJ′ or TRBJ′, for the alpha and beta chain respectively, and for the beta chain, a diversity or D gene termed TRBD′ (Folch and Lefranc, (2000), Exp Clin Immunogenet 17(2): 107-114; Scaviner and Lefranc, (2000), Exp Clin Immunogenet 17(2): 97-106; LeFranc and LeFranc, (2001), “T cell Receptor Factsbook”, Academic Press). The huge diversity of alpha and beta variable region sequences results from combinatorial rearrangements between the various V, J and D genes, which include allelic variants, and additional junctional diversity (Arstila, et al., (1999), Science 286(5441): 958-961; Robins et al., (2009), Blood 114(19): 4099-4107.) The constant, or C, regions of TCR alpha and beta chains are referred to as TRAC and TRBC respectively (Lefranc, (2001), Curr Protoc Immunol Appendix 1: Appendix 10).

The TCR sequences defined herein are described with reference to IMGT nomenclature which is widely known and accessible to those working in the TCR field. For example, see: LeFranc and LeFranc, (2001). “T cell Receptor Factsbook”, Academic Press; Lefranc, (2011), Cold Spring Harb Protoc 2011 (6): 595-603; Lefranc, (2001), Curr Protoc Immunol Appendix 1: Appendix 10; and Lefranc, (2003), Leukemia 17(1): 260-266. αβ TCRs consist of two disulfide linked chains. Each chain (alpha and beta) is generally regarded as having two domains, namely a variable and a constant domain. A short joining region connects the variable and constant domains and is typically considered part of the variable region. Additionally, the beta chain usually contains a short diversity region between the variable and joining regions.

Provided herein are T-cell receptor (TCR) alpha and beta chain pairs that bind the MAGE-A4 derived peptides GVYDGREHTV (SEQ ID NO:1) or KVLEHVVRV (SEQ ID NO: 2) when presented by an HLA class I molecule. In some embodiments, the HLA class I molecule is HLA-A*02:01. The identification of particular TCR sequences that bind to GVYDGREHTV HLA-A*02:01 or KVLEHVVRV HLA-A*02:01 complex is advantageous for the development of novel immunotherapies.

“TCR alpha and beta chain pair” may also be referred to herein as “TCR,” “a TCR,” or “the TCR.” When expressed recombinantly in a cell, e.g., a T cell, the TCR binds to the MAGEA4 peptide-HLA complex on a cell, e.g., a cancer cell, and such binding leads to activation of the recombinant cell. Activation of the T cell leads to the death or destruction of the cancer cell. Methods of determining T-cell activation are known in the art and provided with the Examples herein.

In preferred embodiments, the potency or cytolytic activity (cytotoxicity) of a recombinant cell of the present invention is defined by (1) 80-100% lysis of HLA-A*02:01 target cells loaded with peptide at ˜100 copies (˜10⁻⁸ M) per cell in a T cell dependent cellular cytotoxicity (TDCC) assay, T2/peptide loading assay or (2) 80-100% lysis of natural pMHC target-positive cancer cell lines.

Each TCR alpha and beta chain comprises variable and constant domains. Within the variable domain (Vα or Vβ) are three CDRs (complementarity determining regions): CDR1, CDR2, and CDR3. The various alpha and beta chains variable domains are distinguishable by their framework along with their CDR1, CDR2, and part of their CDR3 sequences.

In the present specification and claims, the term “TCR alpha (or a) variable domain” refers to the concatenation of TRAV and TRAJ regions; a TRAV region only; or TRAV and a partial TRAJ region, and the term TCR alpha (or a) constant domain refers to the extracellular TRAC region, or to a C-terminal truncated or full length TRAC sequence. Likewise the term “TCR beta (or β) variable domain” may refer to the concatenation of TRBV and TRBD/TRBJ regions; to the TRBV and TRBD regions only; to the TRBV and TRBJ regions only; or to the TRBV and partial TRBD and/or TRBJ regions, and the term TCR beta (or β) constant domain refers to the extracellular TRBC region, or to a C-terminal truncated or full length TRBC sequence.

In preferred embodiments, the TCR comprises an alpha chain having a CDR3 set forth in SEQ ID Nos: 7, 17, 27, 37, 47, 57, 67, or 77 and a beta chain having a CDR3 set forth in SEQ ID Nos: 8, 18, 28, 38, 48, 58, 68, or 78. The CDR3 region may be determined by commercially available software (e.g. Cellranger; 10× Genomics). The TCR alpha chain may comprise a sequence at least 95%, at least 96%, at least 97%, at least 98%, at least 99%, or 100% identical to the sequence set forth in any of SEQ ID Nos: 9, 10, 19, 20, 29, 30, 39, 40, 49, 50, 59, 60, 69, 70, 79, or 80. The TCR beta chain may comprise a sequence at least 95%, at least 96%, at least 97%, at least 98%, at least 99%, or 100% identical to the sequence set forth in any of SEQ ID Nos: 11, 12, 21, 22, 31, 32, 41, 42, 51, 52, 61, 62, 71, 72, 81, or 82. Methods of determining the identity between two sequences are well-known in the art, e.g., BLAST or Geneious. In certain embodiments, the C-terminal or N-terminal 1, 2, 3, 4, 5, 6, 7, 8, 9, or 10 residues of any of the sequences set forth is any of SEQ ID Nos: 9, 10, 19, 20, 29, 30, 39, 40, 49, 50, 59, 60, 69, 70, 79, or 80 or any of the sequences set forth in any of SEQ ID Nos: 11, 12, 21, 22, 31, 32, 41, 42, 51, 52, 61, 62, 71, 72, 81, or 82 may be truncated or removed. Exemplary TCRs and the corresponding alpha and beta chain CDR3 and full-length SEQ ID Nos. are provided in Table 1A and Table 1B.

In one embodiment, a TCR1 alpha chain comprises a TRAV4*01 and TRAJ9*01 variable region chain usage. In one embodiment, a TCR1 beta chain comprises a TRBV11-2*01, TRBD2*02, and TRBJ1-4*01 variable region chain usage and a TRBC1*01 constant region chain usage. In one embodiment, a TCR1 comprises a TCR1 alpha chain comprising a TRAV4*01 and TRAJ9*01 variable region chain usage and a TCR1 beta chain comprising a TRBV11-2*01, TRBD2*02, and TRBJ1-4*01 variable region chain usage and a TRBC1*01 constant region chain usage.

In one embodiment, a TCR2 alpha chain comprises a TRAV8-1*01 and TRAJ37*01 variable region chain usage. In one embodiment, a TCR2 beta chain comprises a TRBV2*01, TRBD1*01, and TRBJ2-7*01 variable region chain usage and a TRBC2*01 constant region chain usage. In one embodiment, a TCR2 comprises a TCR2 alpha chain comprising a TRAV8-1*01 and TRAJ37*01 variable region chain usage and a TCR2 beta chain comprising a TRBV2*01, TRBD1*01, and TRBJ2-7*01 variable region chain usage and a TRBC2*01 constant region chain usage.

In one embodiment, a TCR3 alpha chain comprises a TRAV13-2*01 and TRAJ5*01 variable region chain usage. In one embodiment, a TCR3 beta chain comprises a TRBV5-6*01, TRBD1*01, and TRBJ2-2*01 variable region chain usage and a TRBC2*01 constant region chain usage. In one embodiment, a TCR3 comprises a TCR3 alpha chain comprising a TRAV13-2*01 and TRAJ5*01 variable region chain usage and a TCR3 beta chain comprising a TRBV5-6*01, TRBD1*01, and TRBJ2-2*01 variable region chain usage and a TRBC2*01 constant region chain usage.

In one embodiment, a TCR4 alpha chain comprises a TRAV4*01 and TRAJ43*01 variable region chain usage. In one embodiment, a TCR4 beta chain comprises a TRBV11-2*01 and TRBJ2-7*01 variable region chain usage and a TRBC2*01 constant region chain usage. In one embodiment, a TCR4 comprises a TCR4 alpha chain comprising a TRAV4*01 and TRAJ43*01 variable region chain usage and a TCR4 beta chain comprising a TRBV11-2*01 and TRBJ2-7*01 variable region chain usage and a TRBC2*01 constant region chain usage.

In one embodiment, a TCR5 alpha chain comprises a TRAV4*01 and TRAJ9*01 variable region chain usage. In one embodiment, a TCR5 beta chain comprises a TRBV11-2*01, TRBD2*02, and TRBJ1-1*01 variable region chain usage and a TRBC1*01 constant region chain usage. In one embodiment, a TCR5 comprises a TCR5 alpha chain comprising a TRAV4*01 and TRAJ9*01 variable region chain usage and a TCR5 beta chain comprising a TRBV11-2*01, TRBD2*02, and TRBJ1-1*01 variable region chain usage and a TRBC1*01 constant region chain usage.

In one embodiment, a TCR6 alpha chain comprises a TRAV38-1*01 and TRAJ41*01 variable region chain usage. In one embodiment, a TCR6 beta chain comprises a TRBV28*01, TRBD1*01, and TRBJ2-3*01 variable region chain usage and a TRBC2*01 constant region chain usage. In one embodiment, a TCR6 comprises a TCR6 alpha chain comprising a TRAV38-1*01 and TRAJ41*01 variable region chain usage and a TCR6 beta chain comprising a TRBV28*01, TRBD1*01, and TRBJ2-3*01 variable region chain usage and a TRBC2*01 constant region chain usage.

In one embodiment, a TCR7 alpha chain comprises a TRAV38-1*01 and TRAJ29*01 variable region chain usage. In one embodiment, a TCR7 beta chain comprises a TRBV6-6*02 and TRBJ2-1*01 variable region chain usage and a TRBC2*01 constant region chain usage. In one embodiment, a TCR7 comprises a TCR7 alpha chain comprising a TRAV38-1*01 and TRAJ29*01 variable region chain usage and a TCR7 beta chain comprising a TRBV6-6*02 and TRBJ2-1*01 variable region chain usage and a TRBC2*01 constant region chain usage.

In one embodiment, a TCR8 alpha chain comprises a TRAV21*01 and TRAJ31*01 variable region chain usage. In one embodiment, a TCR8 beta chain comprises a TRBV2*01, TRBD2*01, and TRBJ2-7*01 variable region chain usage and a TRBC2*01 constant region chain usage. In one embodiment, a TCR8 comprises a TCR8 alpha chain comprising a TRAV21*01 and TRAJ31*01 variable region chain usage and a TCR8 beta chain comprising a TRBV2*01, TRBD2*01, and TRBJ2-7*01 variable region chain usage and a TRBC2*01 constant region chain usage.

In certain embodiments, the variable domain of a TCR alpha or beta chain may be fused to a non-TCR polypeptide. The exemplary alpha and beta chain variable domains may be used to create a soluble TCR capable of binding the MAGE-A4 derived peptide in the context of an HLA molecule.

The TCR of the invention may be an alpha-beta heterodimer, having an alpha chain TRAC constant domain sequence and a beta chain TRBC1 or TRBC2 constant domain sequence. The alpha and beta chain constant domain sequences may be modified by truncation or substitution to delete the native disulfide bond between Cys4 of exon 2 of TRAC and Cys2 of exon 2 of TRBC1 or TRBC2 and/or the alpha and/or beta chain constant domain sequence(s) may be modified by substitution of cysteine residues to form a non-native disulfide bond between the alpha and beta constant domains of the TCR; for example, substitution of Thr 48 of TRAC and Ser 57 of TRBC1 or TRBC2 to cysteines which form a non-native disulfide bond.

The TCR of the invention may be in single chain format of the type Va-L-νβ, νβ-L-V, Va-Ca-L-νβ, wherein V and νβ are TCR and β variable regions respectively, C and are TCR and β constant regions respectively, and L is a linker sequence. The soluble TCRs may be in single chain format wherein the alpha and beta variable domains are connected by a linker. The soluble TCRs may be fused or connected to a therapeutic or imaging agent.

The TCRs of the present invention may also include one or more conservative substitutions which have a similar amino acid sequence and/or which retain the same function. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide. Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulfur containing side chains). Substitutions of this nature are often referred to as “conservative” or “semi-conservative” amino acid substitutions. The present invention therefore extends to use of a TCR comprising an amino acid sequence described above but with one or more conservative substitutions in the sequence.

Exemplary TCRs and the corresponding sequences are provided in Table 1a and 1b, respectively.

TABLE 1A

| | | | | | | | | Alpha | Beta |
|---|---|---|---|---|---|---|---|---|---|
| | | | | | | | | mature | mature |
| | | Alpha | Beta | Alpha | Alpha | Alpha | Beta | full- | full- |
| | | CDR1 | CDR1 | CDR2 | CDR2 | CDR3 | CDR3 | length | length |
| | | SEQ | SEQ | SEQ | SEQ | SEQ | SEQ | SEQ | SEQ |
| | Peptide | ID | ID | ID | ID | ID | ID | ID | ID |
| TCR | target | NO: | NO: | NO: | NO: | NO: | NO: | NO: | NO: |
| 1 | SEQ ID | 3 | 4 | 5 | 6 | 7 | 8 | 9 | 11 |
| | NO: 1 | | | | | | | | |
| | (TCR23) | | | | | | | | |
| 2 | SEQ ID | 13 | 14 | 15 | 16 | 17 | 18 | 19 | 21 |
| | NO: 1 | | | | | | | | |
| | (TCR24) | | | | | | | | |
| 3 | SEQ ID | 23 | 24 | 25 | 26 | 27 | 28 | 29 | 31 |
| | NO: 2 | | | | | | | | |
| | (TCR2) | | | | | | | | |
| 4 | SEQ ID | 33 | 34 | 35 | 36 | 37 | 38 | 39 | 41 |
| | NO: 2 | | | | | | | | |
| | (TCR10) | | | | | | | | |
| 5 | SEQ ID | 43 | 44 | 45 | 46 | 47 | 48 | 49 | 51 |
| | NO: 2 | | | | | | | | |
| | (TCR3) | | | | | | | | |
| 6 | SEQ ID | 53 | 54 | 55 | 56 | 57 | 58 | 59 | 61 |
| | NO: 2 | | | | | | | | |
| | (TCR7) | | | | | | | | |
| 7 | SEQ ID | 63 | 64 | 65 | 66 | 67 | 68 | 69 | 71 |
| | NO: 1 | | | | | | | | |
| | (TCR18) | | | | | | | | |
| 8 | SEQ ID | 73 | 74 | 75 | 76 | 77 | 78 | 79 | 81 |
| | NO: 1 | | | | | | | | |
| | (TCR18) | | | | | | | | |

TABLE 1B

| SEQ | | |
|---|---|---|
| ID | | |
| NO: | Description | Sequence |
| 1 | MAGEA4- | GVYDGREHTV |
| | derived | |
| | peptide #1 | |
| 2 | MAGEA4- | KVLEHVVRV |
| | derived | |
| | peptide #2 | |
| 3 | TCR1 alpha | NIATNDY |
| | chain CDR1 | |
| 4 | TCR1 beta | SGHAT |
| | chain CDR1 | |
| 5 | TCR1 alpha | GYKTK |
| | chain CDR2 | |
| 6 | TCR1 beta | FQNNGV |
| | chain CDR2 | |
| 7 | TCR1 alpha | CLVGGFYTGGFKTIF |
| | chain CDR3 | |
| 8 | TCR1 beta | CASSIRDNDEKLFF |
| | chain CDR3 | |
| 9 | TCR1 alpha | LAKTTQPISMDSYEGQEVNITCSHNNIATNDYITWYQQFPSQ |
| | chain mature | GPRFIIQGYKTKVTNEVASLFIPADRKSSTLSLPRVSLSDTAV |
| | peptide | YYCLVGGFYTGGFKTIFGAGTRLFVKANI |
| | sequence | |
| 10 | TCR1 alpha | MRQVARVIVFLTLSTLSLAKTTQPISMDSYEGQEVNITCSHN |
| | chain mature | NIATNDYITWYQQFPSQGPRFIIQGYKTKVTNEVASLFIPAD |
| | peptide | RKSSTLSLPRVSLSDTAVYYCLVGGFYTGGFKTIFGAGTRLF |
| | sequence with | VKANI |
| | signaling | |
| | peptide | |
| 11 | TCR1 beta | EAGVAQSPRYKIIEKRQSVAFWCNPISGHATLYWYQQILGQ |
| | chain mature | GPKLLIQFQNNGVVDDSQLPKDRFSAERLKGVDSTLKIQPA |
| | peptide | KLEDSAVYLCASSIRDNDEKLFFGSGTQLSVLEDLNKVFPPE |
| | sequence | VAVFEPSEAEISHTQKATLVCLATGFFPDHVELSWWVNGKE |
| | | VHSGVSTDPQPLKEQPALNDSRYCLSSRLRVSATFWQNPRN |
| | | HFRCQVQFYGLSENDEWTQDRAKPVTQIVSAEAWGRADCG |
| | | FTSVSYQQGVLSATILYEILLGKATLYAVLVSALVLMAMVK |
| | | RKDF |
| 12 | TCR1 beta | MGTRLLCWAALCLLGAELTEAGVAQSPRYKIIEKRQSVAF |
| | chain mature | WCNPISGHATLYWYQQILGQGPKLLIQFQNNGVVDDSQLPK |
| | peptide | DRFSAERLKGVDSTLKIQPAKLEDSAVYLCASSIRDNDEKLF |
| | sequence with | FGSGTQLSVLEDLNKVFPPEVAVFEPSEAEISHTQKATLVCL |
| | signaling | ATGFFPDHVELSWWVNGKEVHSGVSTDPQPLKEQPALNDS |
| | peptide | RYCLSSRLRVSATFWQNPRNHFRCQVQFYGLSENDEWTQD |
| | | RAKPVTQIVSAEAWGRADCGFTSVSYQQGVLSATILYEILL |
| | | GKATLYAVLVSALVLMAMVKRKDF |
| 13 | TCR2 alpha | YGGTVN |
| | chain CDR1 | |
| 14 | TCR2 beta | SNHLY |
| | chain CDR1 | |
| 15 | TCR2 alpha | YFSGDPLV |
| | chain CDR2 | |
| 16 | TCR2 beta | FYNNEI |
| | chain CDR2 | |
| 17 | TCR2 alpha | CAVGSGNTGKLIF |
| | chain CDR3 | |
| 18 | TCR2 beta | CAYDRDGYEQYF |
| | chain CDR3 | |
| 19 | TCR2 alpha | AQSVSQHNHHVILSEAASLELGCNYSYGGTVNLFWYVQYP |
| | chain mature | GQHLQLLLKYFSGDPLVKGIKGFEAEFIKSKFSFNLRKPS |
| | peptide | VQWSDTAEYFCAVGSGNTGKLIFGQGTTLQVKPDI |
| | sequence | |
| 20 | TCR2 alpha | MLLLLIPVLGMIFALRDARAQSVSQHNHHVILSEAASLELG |
| | chain peptide | CNYSYGGTVNLFWYVQYPGQHLQLLLKYFSGDPLVKGIKGF |
| | sequence with | EAEFIKSKFSFNLRKPSVQWSDTAEYFCAVGSGNTGKLIFG |
| | signaling | QGTTLQVKPDI |
| | peptide | |
| 21 | TCR2 beta | EPEVTQTPSHQVTQMGQEVILRCVPISNHLYFYWYRQILGQ |
| | chain mature | KVEFLVSFYNNEISEKSEIFDDQFSVERPDGSNFTLKIRSTK |
| | peptide | LEDSAMYFCAYDRDGYEQYFGPGTRLTVTEDLKNVFPPEVA |
| | sequence | VFEPSEAEISHTQKATLVCLATGFYPDHVELSWWVNGKEV |
| | | HSGVSTDPQPLKEQPALNDSRYCLSSRLRVSATFWQNPRNH |
| | | FRCQVQFYGLSENDEWTQDRAKPVTQIVSAEAWGRADCGF |
| | | TSESYQQGVLSATILYEILLGKATLYAVLVSALVLMAMVKR |
| | | KDSRG |
| 22 | TCR2 beta | MDTWLVCWAIFSLLKAGLTEPEVTQTPSHQVTQMGQEVILR |
| | chain peptide | CVPISNHLYFYWYRQILGQKVEFLVSFYNNEISEKSEIFDD |
| | sequence with | QFSVERPDGSNFTLKIRSTKLEDSAMYFCAYDRDGYEQYFG |
| | signaling | PGTRLTVTEDLKNVFPPEVAVFEPSEAEISHTQKATLVCLAT |
| | peptide | GFYPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSESYQQGVLSATILYEILLGKA |
| | | TLYAVLVSALVLMAMVKRKDSRG |
| 23 | TCR3 alpha | NSASDY |
| | chain CDR1 | |
| 24 | TCR3 beta | SGHDT |
| | chain CDR1 | |
| 25 | TCR3 alpha | IRSNMDK |
| | chain CDR2 | |
| 26 | TCR3 beta | YYEEEE |
| | chain CDR2 | |
| 27 | TCR3 alpha | CAEASPRQDTGRRALTF |
| | chain CDR3 | |
| 28 | TCR3 beta | CASSLWTGSGELFF |
| | chain CDR3 | |
| 29 | TCR3 alpha | GESVGLHLPTLSVQEGDNSIINCAYSNSASDYFIWYKQESGK |
| | chain mature | GPQFIIDIRSNMDKRQGQRVTVLLNKTVKHLSLQIAATQPGD |
| | peptide | SAVYFCAEASPRQDTGRRALTFGSGTRLQVQPNI |
| | sequence | |
| 30 | TCR3 alpha | MAGIRALFMYLWLQLDWVSRGESVGLHLPTLSVQEGDNSII |
| | chain peptide | NCAYSNSASDYFIWYKQESGKGPQFIIDIRSNMDKRQGQRV |
| | sequence with | TVLLNKTVKHLSLQIAATQPGDSAVYFCAEASPRQDTGRRA |
| | signaling | LTFGSGTRLQVQPNI |
| | peptide | |
| 31 | TCR3 beta | DAGVTQSPTHLIKTRGQQVTLRCSPKSGHDTVSWYQQALG |
| | chain mature | QGPQFIFQYYEEEERQRGNFPDRFSGHQFPNYSSELNVNALL |
| | peptide | LGDSALYLCASSLWTGSGELFFGEGSRLTVLEDLKNVFPPE |
| | sequence | VAVFEPSEAEISHTQKATLVCLATGFYPDHVELSWWVNGK |
| | | EVHSGVSTDPQPLKEQPALNDSRYCLSSRLRVSATFWQNPR |
| | | NHFRCQVQFYGLSENDEWTQDRAKPVTQIVSAEAWGRADC |
| | | GFTSESYQQGVLSATILYEILLGKATLYAVLVSALVLMAMV |
| | | KRKDSRG |
| 32 | TCR3 beta | MGPGLLCWALLCLLGAGLVDAGVTQSPTHLIKTRGQQVTL |
| | chain peptide | RCSPKSGHDTVSWYQQALGQGPQFIFQYYEEEERQRGNFPD |
| | sequence with | RFSGHQFPNYSSELNVNALLLGDSALYLCASSLWTGSGELF |
| | signaling | FGEGSRLTVLEDLKNVFPPEVAVFEPSEAEISHTQKATLVCL |
| | peptide | ATGFYPDHVELSWWVNGKEVHSGVSTDPQPLKEQPALNDS |
| | | RYCLSSRLRVSATFWQNPRNHFRCQVQFYGLSENDEWTQDR |
| | | AKPVTQIVSAEAWGRADCGFTSESYQQGVLSATILYEILLG |
| | | KATLYAVLVSALVLMAMVKRKDSRG |
| 33 | TCR4 alpha | NIATNDY |
| | chain CDR1 | |
| 34 | TCR4 beta | SGHAT |
| | chain CDR1 | |
| 35 | TCR4 alpha | GYKTK |
| | chain CDR2 | |
| 36 | TCR4 beta | FQNNGV |
| | chain CDR2 | |
| 37 | TCR4 alpha | CLVGGDEDMRF |
| | chain CDR3 | |
| 38 | TCR4 beta | CASSLEYGPTYEQYF |
| | chain CDR3 | |
| 39 | TCR4 alpha | LAKTTQPISMDSYEGQEVNITCSHNNIATNDYITWYQQFPSQ |
| | chain mature | GPRFIIQGYKTKVTNEVASLFIPADRKSSTLSLPRVSLSDTAV |
| | peptide | YYCLVGGDEDMRFGAGTRLTVKPNI |
| | sequence | |
| 40 | TCR4 alpha | MRQVARVIVELTLSTLSLAKTTQPISMDSYEGQEVNITCSHN |
| | chain peptide | NIATNDYITWYQQFPSQGPRFIIQGYKTKVTNEVASLFIPAD |
| | sequence with | RKSSTLSLPRVSLSDTAVYYCLVGGDEDMRFGAGTRLTVKP |
| | signaling | NI |
| | peptide | |
| 41 | TCR4 beta | EAGVAQSPRYKIIEKRQSVAFWCNPISGHATLYWYQQILGQ |
| | chain mature | GPKLLIQFQNNGVVDDSQLPKDRFSAERLKGVDSTLKIQPA |
| | peptide | KLEDSAVYLCASSLEYGPTYEQYFGPGTRLTVTEDLKNVFP |
| | sequence | PEVAVFEPSEAEISHTQKATLVCLATGFYPDHVELSWWVNG |
| | | KEVHSGVSTDPQPLKEQPALNDSRYCLSSRLRVSATFWQNP |
| | | RNHFRCQVQFYGLSENDEWTQDRAKPVTQIVSAEAWGRAD |
| | | CGFTSESYQQGVLSATILYEILLGKATLYAVLVSALVLMAM |
| | | VKRKDSRG |
| 42 | TCR4 beta | MGTRLLCWAALCLLGAELTEAGVAQSPRYKIIEKRQSVAF |
| | chain peptide | WCNPISGHATLYWYQQILGQGPKLLIQFQNNGVVDDSQLPK |
| | sequence with | DRFSAERLKGVDSTLKIQPAKLEDSAVYLCASSLEYGPTYE |
| | signaling | QYFGPGTRLTVTEDLKNVFPPEVAVFEPSEAEISHTQKATLV |
| | peptide | CLATGFYPDHVELSWWVNGKEVHSGVSTDPQPLKEQPALN |
| | | DSRYCLSSRLRVSATFWQNPRNHFRCQVQFYGLSENDEWT |
| | | QDRAKPVTQIVSAEAWGRADCGFTSESYQQGVLSATILYEI |
| | | LLGKATLYAVLVSALVLMAMVKRKDSRG |
| 43 | TCR5 alpha | ATGYPS |
| | chain CDR1 | |
| 44 | TCR5 beta | SGHVS |
| | chain CDR1 | |
| 45 | TCR5 alpha | ATKADDK |
| | chain CDR2 | |
| 46 | TCR5 beta | FNYEAQ |
| | chain CDR2 | |
| 47 | TCR5 alpha | CALSVDGQKLLF |
| | chain CDR3 | |
| 48 | TCR5 beta | CASSLADTEAFF |
| | chain CDR3 | |
| 49 | TCR5 alpha | GDSVTQMEGPVTLSEEAFLTINCTYTATGYPSLFWYVQYPG |
| | chain mature | EGLQLLLKATKADDKGSNKGFEATYRKETTSFHLEKGSVQ |
| | peptide | VSDSAVYFCALSVDGQKLLFARGTMLKVDLNI |
| | sequence | |
| 50 | TCR5 alpha | MNYSPGLVSLILLLLGRTRGDSVTQMEGPVTLSEEAFLTINC |
| | chain peptide | TYTATGYPSLFWYVQYPGEGLQLLLKATKADDKGSNKGFE |
| | sequence with | ATYRKETTSFHLEKGSVQVSDSAVYFCALSVDGQKLLFARG |
| | signaling | TMLKVDLNI |
| | peptide | |
| 51 | TCR5 beta | GAGVSQSPRYKVTKRGQDVALRCDPISGHVSLYWYRQALG |
| | chain mature | QGPEFLTYFNYEAQQDKSGLPNDRFSAERPEGSISTLTIQRTE |
| | peptide | QRDSAMYRCASSLADTEAFFGQGTRLTVVEDLNKVFPPEV |
| | sequence | AVFEPSEAEISHTQKATLVCLATGFFPDHVELSWWVNGKEV |
| | | HSGVSTDPQPLKEQPALNDSRYCLSSRLRVSATFWQNPRNH |
| | | FRCQVQFYGLSENDEWTQDRAKPVTQIVSAEAWGRADCGF |
| | | TSVSYQQGVLSATILYEILLGKATLYAVLVSALVLMAMVKR |
| | | KDF |
| 52 | TCR5 beta | MGTSLLCWVVLGFLGTDHTGAGVSQSPRYKVTKRGQDVA |
| | chain peptide | LRCDPISGHVSLYWYRQALGQGPEFLTYFNYEAQQDKSGLP |
| | sequence with | NDRFSAERPEGSISTLTIQRTEQRDSAMYRCASSLADTEAFF |
| | signaling | GQGTRLTVVEDLNKVFPPEVAVFEPSEAEISHTQKATLVCL |
| | peptide | ATGFFPDHVELSWWVNGKEVHSGVSTDPQPLKEQPALNDS |
| | | RYCLSSRLRVSATFWQNPRNHFRCQVQFYGLSENDEWTQD |
| | | RAKPVTQIVSAEAWGRADCGFTSVSYQQGVLSATILYEILL |
| | | GKATLYAVLVSALVLMAMVKRKDF |
| 53 | TCR6 alpha | TSENNYY |
| | chain CDR1 | |
| 54 | TCR6 beta | MDHEN |
| | chain CDR1 | |
| 55 | TCR6 alpha | QEAYKQQN |
| | chain CDR2 | |
| 56 | TCR6 beta | SYDVKM |
| | chain CDR2 | |
| 57 | TCR6 alpha | CAFVDSGYALNF |
| | chain CDR3 | |
| 58 | TCR6 beta | CASSLDGARTQYF |
| | chain CDR3 | |
| 59 | TCR6 alpha | AQTVTQSQPEMSVQEAETVTLSCTYDTSENNYYLFWYKQP |
| | chain mature | PSRQMILVIRQEAYKQQNATENRFSVNFQKAAKSFSLKISDS |
| | peptide | QLGDTAMYFCAFVDSGYALNFGKGTSLLVTPHI |
| | sequence | |
| 60 | TCR6 alpha | MTRVSLLWAVVVSTCLESGMAQTVTQSQPEMSVQEAETVT |
| | chain peptide | LSCTYDTSENNYYLFWYKQPPSRQMILVIRQEAYKQQNATE |
| | sequence with | NRFSVNFQKAAKSFSLKISDSQLGDTAMYFCAFVDSGYALN |
| | signaling | FGKGTSLLVTPHI |
| | peptide | |
| 61 | TCR6 beta | DVKVTQSSRYLVKRTGEKVFLECVQDMDHENMFWYRQDP |
| | chain mature | GLGLRLIYFSYDVKMKEKGDIPEGYSVSREKKERFSLILESA |
| | peptide | STNQTSMYLCASSLDGARTQYFGPGTRLTVLEDLKNVFPPE |
| | sequence | VAVFEPSEAEISHTQKATLVCLATGFYPDHVELSWWVNGK |
| | | EVHSGVSTDPQPLKEQPALNDSRYCLSSRLRVSATFWQNPR |
| | | NHFRCQVQFYGLSENDEWTQDRAKPVTQIVSAEAWGRADC |
| | | GFTSESYQQGVLSATILYEILLGKATLYAVLVSALVLMAMV |
| | | KRKDSRG |
| 62 | TCR6 beta | MGIRLLCRVAFCFLAVGLVDVKVTQSSRYLVKRTGEKVFLE |
| | chain peptide | CVQDMDHENMFWYRQDPGLGLRLIYFSYDVKMKEKGDIPE |
| | sequence with | GYSVSREKKERFSLILESASTNQTSMYLCASSLDGARTQYFG |
| | signaling | PGTRLTVLEDLKNVFPPEVAVFEPSEAEISHTQKATLVCLAT |
| | peptide | GFYPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSESYQQGVLSATILYEILLGKA |
| | | TLYAVLVSALVLMAMVKRKDSRG |
| 63 | TCR7 alpha | TSENNYY |
| | chain CDR1 | |
| 64 | TCR7 beta | MNHNY |
| | chain CDR1 | |
| 65 | TCR7 alpha | QEAYKQQN |
| | chain CDR2 | |
| 66 | TCR7 beta | SVGAGI |
| | chain CDR2 | |
| 67 | TCR7 alpha | CALLDSGNTPLVF |
| | chain CDR3 | |
| 68 | TCR7 beta | CASSYTNNEQFF |
| | chain CDR3 | |
| 69 | TCR7 alpha | AQTVTQSQPEMSVQEAETVTLSCTYDTSENNYYLFWYKQP |
| | chain mature | PSRQMILVIRQEAYKQQNATENRFSVNFQKAAKSFSLKISDS |
| | peptide | QLGDTAMYFCALLDSGNTPLVFGKGTRLSVIANIQNPDPAV |
| | sequence | YQLRDSKSSDKSVCLFTDFDSQTNVSQSKDSDVYITDKTVL |
| | | DMRSMDFKSNSAVAWSNKSDFACANAFNNSIIPEDTFFPSPE |
| | | SSCDVKLVEKSFETDTNLNFQNLSVIGFRILLLKVAGFNLLM |
| | | TLRLWSS |
| 70 | TCR7 alpha | MTRVSLLWAVVVSTCLESGMAQTVTQSQPEMSVQEAETVT |
| | chain peptide | LSCTYDTSENNYYLFWYKQPPSRQMILVIRQEAYKQQNATE |
| | sequence with | NRFSVNFQKAAKSFSLKISDSQLGDTAMYFCALLDSGNTPL |
| | signaling | VFGKGTRLSVIANIQNPDPAVYQLRDSKSSDKSVCLFTDFDS |
| | peptide | QTNVSQSKDSDVYITDKTVLDMRSMDFKSNSAVAWSNKSDF |
| | | ACANAFNNSIIPEDTFFPSPESSCDVKLVEKSFETDTNLNFQ |
| | | NLSVIGFRILLLKVAGFNLLMTLRLWSS |
| 71 | TCR7 beta | NAGVTQTPKFRILKIGQSMTLQCAQDMNHNYMYWYRQDP |
| | chain mature | GMGLKLIYYSVGAGITDKGEVPNGYNVSRSTTEDFPLRLEL |
| | peptide | AAPSQTSVYFCASSYTNNEQFFGPGTRLTVLEDLKNVFPPEV |
| | sequence | AVFEPSEAEISHTQKATLVCLATGFYPDHVELSWWVNGKE |
| | | VHSGVSTDPQPLKEQPALNDSRYCLSSRLRVSATFWQNPRN |
| | | HFRCQVQFYGLSENDEWTQDRAKPVTQIVSAEAWGRADCG |
| | | FTSESYQQGVLSATILYEILLGKATLYAVLVSALVLMAMVK |
| | | RKDSRG |
| 72 | TCR7 beta | MSISLLCCAAFPLLWAGPVNAGVTQTPKFRILKIGQSMTLQ |
| | chain peptide | CAQDMNHNYMYWYRQDPGMGLKLIYYSVGAGITDKGEVPNG |
| | sequence with | YNVSRSTTEDFPLRLELAAPSQTSVYFCASSYTNNEQFFGP |
| | signaling | GTRLTVLEDLKNVFPPEVAVFEPSEAEISHTQKATLVCLAT |
| | peptide | GFYPDHVELSWWVNGKEVHSGVSTDPQPLKEQPALNDSRYC |
| | | LSSRLRVSATFWQNPRNHFRCQVQFYGLSENDEWTQDRAKP |
| | | VTQIVSAEAWGRADCGFTSESYQQGVLSATILYEILLGKAT |
| | | LYAVLVSALVLMAMVKRKDSRG |
| 73 | TCR8 alpha | DSAIYN |
| | chain CDR1 | |
| 74 | TCR8 beta | SNHLY |
| | chain CDR1 | |
| 75 | TCR8 alpha | IQSSQRE |
| | chain CDR2 | |
| 76 | TCR8 beta | FYNNEI |
| | chain CDR2 | |
| 77 | TCR8 alpha | CAVDAHARLMF |
| | chain CDR3 | |
| 78 | TCR8 beta | CASISGEQYF |
| | chain CDR3 | |
| 79 | TCR8 alpha | KQEVTQIPAALSVPEGENLVLNCSFTDSAIYNLQWFRQDPG |
| | chain mature | KGLTSLLLIQSSQREQTSGRLNASLDKSSGRSTLYIAASQPG |
| | peptide | DSATYLCAVDAHARLMFGDGTQLVVKPNI |
| | sequence | |
| 80 | TCR8 alpha | METLLGLLILWLQLQWVSSKQEVTQIPAALSVPEGENLVLN |
| | chain peptide | CSFTDSAIYNLQWFRQDPGKGLTSLLLIQSSQREQTSGRLNA |
| | sequence with | SLDKSSGRSTLYIAASQPGDSATYLCAVDAHARLMFGDGTQ |
| | signaling | LVVKPNI |
| | peptide | |
| 81 | TCR8 beta | EPEVTQTPSHQVTQMGQEVILRCVPISNHLYFYWYRQILGQ |
| | chain mature | KVEFLVSFYNNEISEKSEIFDDQFSVERPDGSNFTLKIRSTKL |
| | peptide | EDSAMYFCASISGEQYFGPGTRLTVTEDLKNVFPPEVAVFEP |
| | sequence | SEAEISHTQKATLVCLATGFYPDHVELSWWVNGKEVHSGV |
| | | STDPQPLKEQPALNDSRYCLSSRLRVSATFWQNPRNHFRCQ |
| | | VQFYGLSENDEWTQDRAKPVTQIVSAEAWGRADCGFTSES |
| | | YQQGVLSATILYEILLGKATLYAVLVSALVLMAMVKRKDS |
| | | RG |
| 82 | TCR8 beta | MDTWLVCWAIFSLLKAGLTEPEVTQTPSHQVTQMGQEVIL |
| | chain peptide | RCVPISNHLYFYWYRQILGQKVEFLVSFYNNEISEKSEIFDD |
| | sequence with | QFSVERPDGSNFTLKIRSTKLEDSAMYFCASISGEQYFGPGT |
| | signaling | RLTVTEDLKNVFPPEVAVFEPSEAEISHTQKATLVCLATGFY |
| | peptide | PDHVELSWWVNGKEVHSGVSTDPQPLKEQPALNDSRYCLS |
| | | SRLRVSATFWQNPRNHFRCQVQFYGLSENDEWTQDRAKPV |
| | | TQIVSAEAWGRADCGFTSESYQQGVLSATILYEILLGKATLY |
| | | AVLVSALVLMAMVKRKDSRG |

The TCR alpha or beta variable domain may comprise a sequence at least 95%, at least 96%, at least 97%, at least 98%, at least 99%, or 100% identical to any of the sequences specified in Table 2. The TCR beta chain may comprise a sequence at least 80%, at least 85%, at least 90%, at least 95%, at least 96%, at least 97%, at least 98%, at least 99%, or 100% identical to the sequence set forth is any of SEQ ID Nos: 46-56. In certain embodiments, the C-terminal or N-terminal 1, 2, 3, 4, 5, 6, 7, 8, 9, or 10 residues of any of the sequences specified in Table 2 and Table 1B SEQ ID NOs: 35-56 may be truncated or removed.

Although recognition of the target peptide in the context of HLA is required for efficacy, for safety purposes, in some embodiments it is preferred that the TCR lacks cross-reactivity with structurally similar peptides when presented by HLA-A*02:01 or with HLA molecules of other allotypes. The cross-reactivity and alloreactivity of the exemplary TCRs described herein are provided in the Examples. Thus, the exemplary TCRs not only are able to recognize the MAGE-A4 peptide in the context of HLA-A*02:01 as expressed on tumor cells and activate a T cell recombinantly expressing the TCR against the tumor cell but also fail to activate or have minimal activation when the recombinant T cell is presented with peptides in the context of HLA-A*02:01 or other HLA molecules that are expressed on normal tissue.

Further embodiments of the present invention include nucleic acids encoding a TCR alpha variable domain, a TCR beta variable domain, or a TCR alpha variable domain and a TCR beta variable domain described herein. In particular embodiments, the nucleic acid encodes one or more of the alpha or beta variable domains set forth in Table 2. In certain embodiments, the nucleic acid encodes both alpha and beta variable domains of TCR1, TCR2, TCR3, TCR4, TCR5, TCR6, TCR7, or TCR8. In preferred embodiments, the nucleic acid encoding the TCR alpha chain variable domain, TCR beta chain variable domain, or TCR alpha chain variable domain and beta chain variable domain is an expression vector wherein the TCR alpha chain variable domain, TCR beta chain variable domain, or TCR alpha chain variable domain and beta chain variable domain is operably linked to a promoter.

The TCR alpha variable domain and beta variable domain may be co-transcribed from the same promoter. For embodiments wherein the alpha variable domain and beta variable domains are linked within a fusion protein, the domains may be co-translated within a single polypeptide as well. In embodiments wherein the alpha domain and beta domain are within separate polypeptides, it is useful to include an internal ribosome entry site (IRES) between the alpha variable domain and beta variable domain coding regions within the expression vector.

Also provided herein are nucleic acids encoding a TCR alpha chain, a TCR beta chain, or a TCR alpha and TCR beta chain described herein. In particular embodiments, the nucleic acid encodes one or more of the alpha or beta chains set forth in Table 1. The encoded alpha or beta chain may be full-length or mature. When mature, i.e., lacking the nature leader sequence associated with that alpha or beta chain, it is preferred that a nucleic acid encoding a signal or leader sequence is operably connected to the nucleic acid encoding the alpha chain or beta chain such that, when translated, the leader sequence directs the alpha or beta chain to the endoplasmic reticulum.

In certain embodiments, the nucleic acid encodes both alpha and beta chains of TCR1, TCR2, TCR3, TCR4, TCR5, TCR6, TCR7, or TCR8. In preferred embodiments, the nucleic acid encoding the TCR alpha chain, TCR beta chain, or TCR alpha chain and beta chain is an expression vector wherein the TCR alpha chain, TCR beta chain, or TCR alpha chain and beta chain is operably linked to a promoter.

The TCR alpha chain and beta chain may be co-transcribed from the same promoter. In such embodiments, it is useful to include an internal ribosome entry site (IRES) between the alpha chain and beta chain coding regions within the expression vector.

The expression vectors of the present invention include, but are not limited to, retroviral or lentiviral vectors. The expression vector may further encode one or more additional proteins besides the TCR alpha chain and/or beta chain. In certain embodiments, the expression vector encodes one or more cytokines. In preferred embodiments, the cytokine is a T cell growth factor such as IL-2, IL-7, IL-12, IL-15, IL-18, or IL-21, along with combinations thereof. Because cytokines can have systemic effects, when the expression vector encoding the cytokine is used to produce a cell for adoptive cell therapy, it is preferred that the cytokine expression is controlled by an inducible promoter. In certain embodiments, the promoter is a composite promoter containing six NFAT (nuclear factor of activated T cells) response elements linked to a minimal IL-2 promoter and the cytokine is IL-12 or a variant thereof. Use of a composite promoter containing six NFAT (nuclear factor of activated T cells) response elements linked to a minimal IL-2 promoter to express IL-12 is described in U.S. Pat. No. 8,556,882.

Provided herein are cells recombinantly expressing an exemplary TCR described herein. Said recombinant cells may comprise one or more expression vectors encoding and expressing a TCR alpha chain, a TCR beta chain, a TCR alpha and beta chain, a TCR alpha variable domain, a TCR beta variable domain, or TCR alpha and beta variable domains. In preferred embodiments, the cell recombinantly expresses TCR1, TCR2, TCR3, TCR4, TCR5, TCR6, TCR7, or TCR8. In certain embodiments, the cell further expresses one or more recombinant cytokines. In preferred embodiments, the cytokine is IL-12 or a variant thereof and said expression is controlled by an inducible promoter, e.g., an NFAT driven promoter.

In certain embodiments, the cells are derived from a sample taken from a cancer patient. Cells, such as T cells or NKT cells, are isolated from the sample and expanded. In certain embodiments, progenitor cells are isolated and matured to the desired cell type. The cells are transfected/transformed with one or more vectors, e.g., lentiviral vectors, encoding the components of the TCR along with any additional polypeptides, e.g., IL-12 or a variant thereof. Such cells may be used for adoptive cell therapy for the cancer patient from whom they were derived.

In other embodiments, a cell line recombinantly expresses a soluble TCR. The soluble TCR may be a fusion protein with an anti-CD3 antigen binding protein such as an scFv.

Provided herein are methods of treating a disease or disorder wherein cells associated with the disease or disorder express MAGE-A4. In preferred embodiments, the cells present the MAGE-A4 derived peptides KVLEHVVRV and/or GVYDGREHTV in the context of an HLA class I molecule, preferably HLA-A2, particularly HLA-A*02:01. Exemplary diseases or disorders that may be treated with the soluble TCRs or recombinant cells of the present invention include hematological or solid tumors. Such diseases and disorders include, but are not limited to, lung cancer, ovarian cancer, squamous cell lung cancer, melanoma, breast cancer, gastric cancer, testicular cancer, head and neck cancer, uterine cancer, esophageal cancer, bladder cancer, and cervical cancer. Preferred diseases and disorders include non-small cell lung cancer (NSCLC), head and neck squamous cell carcinoma (HNSCC), bladder cancer, esophageal cancer, or ovarian cancer.

For certain treatments, a biopsy of the tumor is tested for expression or MAGE-A4. The tumor may also be tested for expression of an appropriate HLA molecule that is recognized by a TCR of the present invention when presenting the MAGE-A4-derived peptide. Patients whose tumors express MAGE-A4 and are of the appropriate HLA haplotype may be administered a soluble TCR or recombinant cell of the present invention.

### EXAMPLES

The following examples, both actual and prophetic, are provided for the purpose of illustrating specific embodiments or features of the present invention and are not intended to limit its scope.

### Example 1—MAGE-A4 is Expressed Across a Broad Range of Solid Tumors with Highly Restricted Normal Tissue Expression

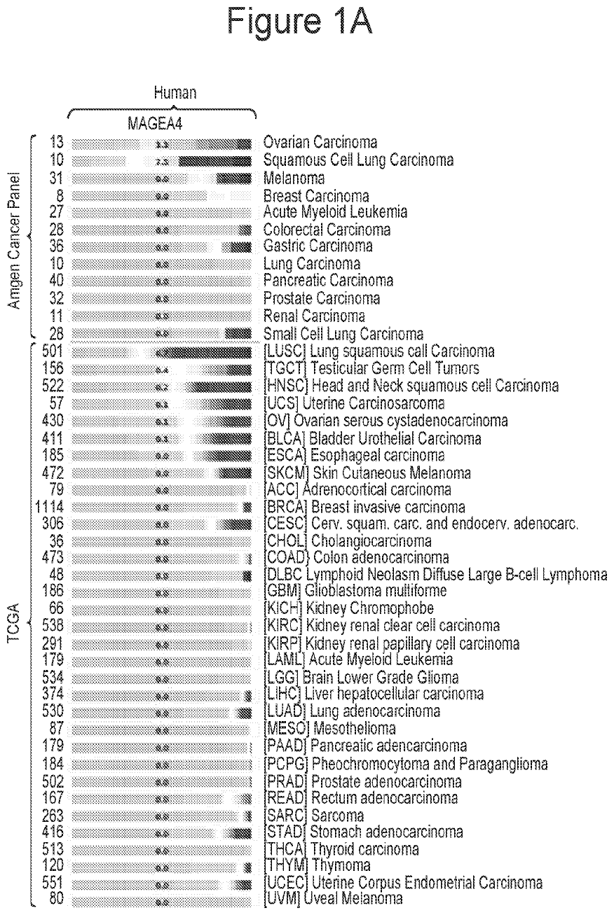

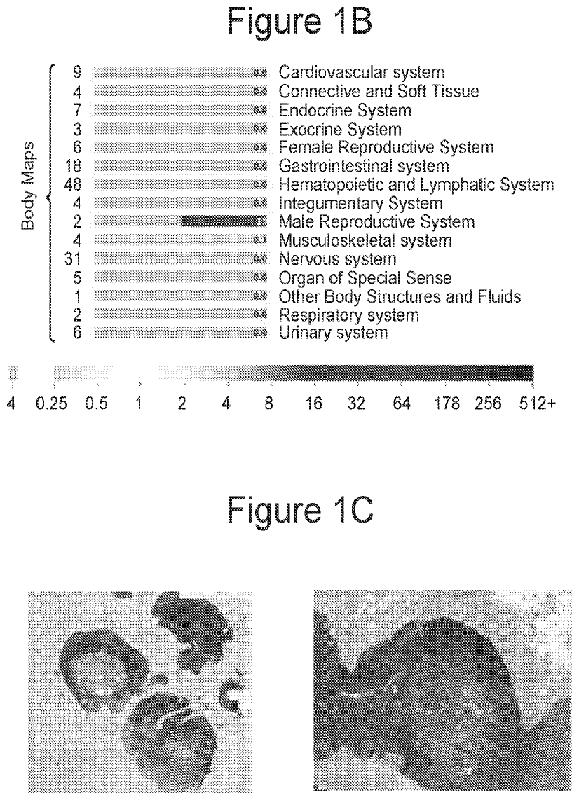

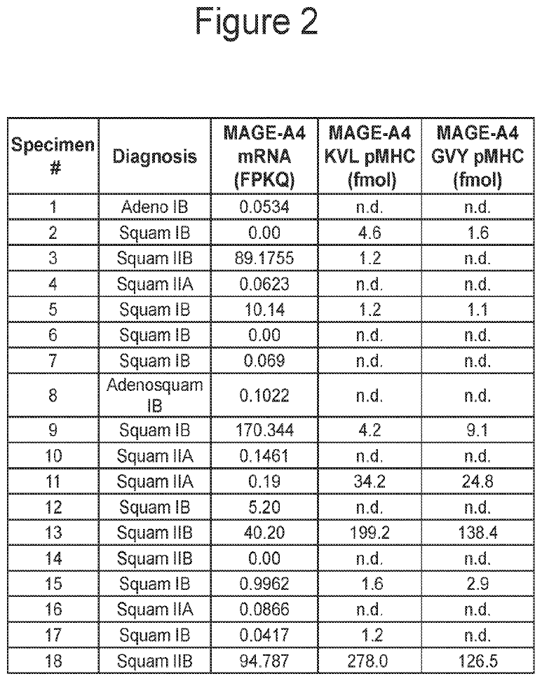

TCGA and applicant's data demonstrate that MAGE-A4 mRNA have high prevalence across a broad range of solid tumors (FIG. 1A). Importantly, Applicant's internal body map data show extremely restricted normal tissue expression of MAGE-A4, except testis, which is an immune privileged site (FIG. 1B). The MAGE-A4 IHC data in NSCLC-squamous (squamous non-small cell lung cancer or lung squamous cell carcinoma) shows within a tumor, MAGE-A4 protein is expressed in the majority of tumor cells (60-100%), and not in stromal cells (FIG. 1C). Presentation of both target MAGE-A4 peptides were identified in U266B1 cells by MS on MHC and confirmed in squamous NSCLC tumors (FIG. 2). The MAGE-A4 peptide GVYDGREHTV (SEQ ID NO: 1) corresponds to amino acid residues 230-239 of the MAGE A4 protein. KVLEHVVRV (SEQ ID NO:2) corresponds to amino acid residues 286-294 of the MAGE A4 protein.

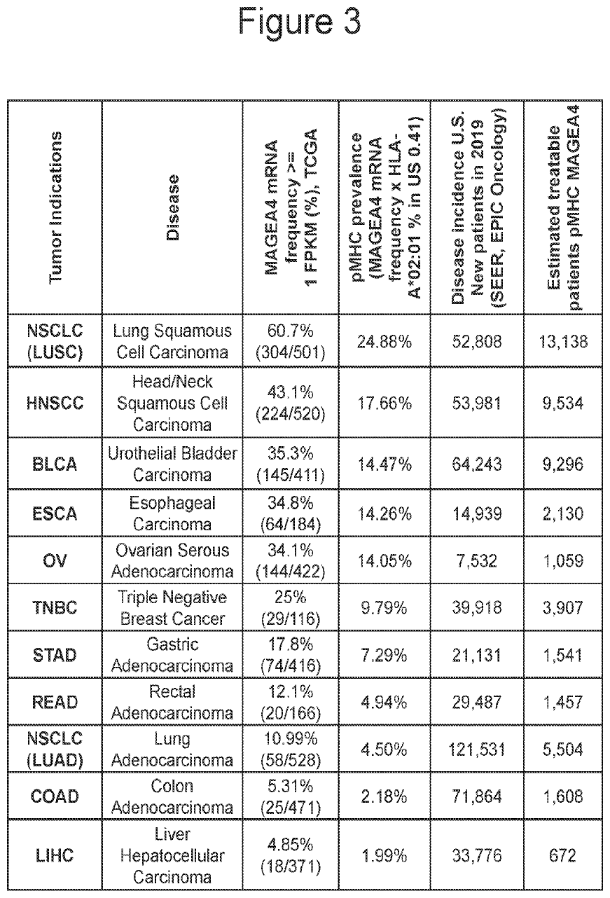

MAGE-A4 is expressed in a wide range of cancer types. The solid tumor indications with MAGE-A4 pMHC expression (MAGE-A4-HLA-A*02:01) include, but are not limited to, ˜24.9% of lung squamous cell carcinoma (NSCLC-squamous, LUSC), ˜17.7% of head and neck squamous cell carcinoma (HNSCC), ˜14.5% of urothelial bladder carcinoma (BLCA), ˜14.3% of esophageal carcinoma, ˜14.1% of ovarian cancer, ˜9.8% of triple negative breast cancer (TNBC), ˜7.3% of gastric cancer (STAD), ˜4.9% of rectal adenocarcinoma (READ), ˜4.5% of lung adenocarcinoma (LUAD), ˜2.2% of colon adenocarcinoma (COAD), and ˜2% of liver hepatocellular carcinoma (LIHC) (FIG. 3). The pMHC target frequency (%) was calculated by MAGE-A4 mRNA expression frequency X HLA-A*02:01 carrier frequency in U.S (0.41). Patient population in specified cancer indication was estimated based on pMHC target frequency (%)×new cases per year in U.S. populations. The TCGA public datasets of RNAseq from tumors of interest were used to estimate MAGE-A4 mRNA expression frequency in each tumor indication at a threshold of MAGEA4>=1 FPKM (FIG. 3). SEER, EPIC Oncology New Patients, or Epiphany/Epic in 2020 was used to estimate disease incidence (new cases per year) in selected tumor indications and hence derive estimated treatable patient population ranges (FIG. 3). HLA-A*02:01 is one of the most common MHC class I allele in U.S. The HLA-A*02:01 haplotype (carrier) frequency estimate in U.S. populations is 0.41 (www.allelefrequencies.net). The largest patient population is in NSCLC-squamous, followed by HNSCC, bladder cancer, esophagus cancer and ovarian cancer (FIG. 3).

### Example 2—Identification of MAGE-A4 PMHC-Specific TCRs

Identification and selection process for lead clinical TCR candidates is outlined in below. First, 101 dominant MAGE-A4 pMHC-specific TCRs targeting MAGEA4 peptide epitopes were identified using 72 healthy HLA-A*02:01+ donors. Using Jurkat activation assays, 10-11 TCR candidates were selected for each target peptide. Based on these TCR sequences, TCR-T cells per donor were generated by transduction of primary pan-T cells isolated from 3 donors with lentivirus carrying individual TCRs. Those TCR-T cells were further evaluated by various functional assays including potency (cytotoxicity) tests with T2 cell line that were pulsed with target peptides and multiple cancer cell lines, a cross-reactivity screen with similar peptides, and an alloreactivity screen. Based on those functional data, we further narrowed down to 2 top TCR candidates. To enhance the in vivo efficacy, all TCRs were manufactured in a TCR-T-IL12 lentiviral construct, where the IL12 payload expression is regulated by TCR activation under a NFAT response element driven promoter. Therefore, only when TCR-T cells bind to the pMHC targets in tumors, the IL12 can be produced.

MAGE-A4 pMHC-Specific TCRs can be Identified from Rare T Cell Clones of Healthy Donor PBMCs

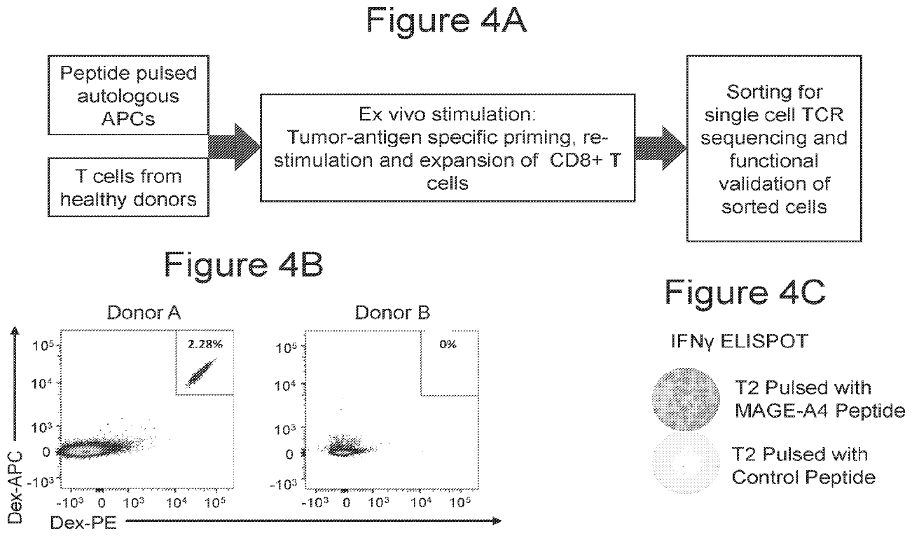

Difficulties in identifying tumor antigen-specific TCRs have hampered the development of TCR-mediated immunotherapies. Despite these challenges, the TCR discovery platform is described herein by which the tumor antigen pMHC-specific TCRs can be identified from rare T cell clones of healthy donors. The frequencies of MAGEA4 pMHC-reactive T cells in PBMCs from healthy HLA-A*02:01+ donors were extremely low, which were typically ˜0% dextramer+ T cells. Dextramer (Dex) is a multimer of peptide-MHC complexes that can specifically bind to TCRs, and therefore can be used to isolate antigen (pMHC)-specific T cells. First, in order to expand the rare tumor antigen-specific T clones, we used 72 healthy HLA-A*02:01+ donor's PBMCs to isolate T cells and autologous antigen presenting cells (APCs) such as monocyte-derived dendritic cells and activated B cells. Upon co-culture of T cells with the autologous APCs pulsed with target peptides, these T cells went through multiple steps of ex vivo stimulations where tumor antigen pMHC-specific priming, restimulation and expansion of pMHC-specific T cells occur. After 3-4 rounds of antigen restimulations, the MAGE-A4 pMHC-specific T cell population was enriched and validated by both dextramer-PE and dextramer-APC stains (FIG. 4). The Dex+ T cells were then sorted for single cell RNAseq to identify the sequences of TCRα and TCRβ chains. Furthermore, those sorted Dex+CD8+ T cells were validated for the antigen-specific IFNγ production by ELISPOT assay using peptide-loaded T2 cells (FIG. 4). This TCR discovery platform led to identification of 101 dominant MAGE-A4 pMHC-specific TCRs from 72 healthy HLA-A*02:01+ donors. Importantly, the TCRs identified from healthy donor blood and have been through thymic natural selection in the human body (in medulla of thymus) to eliminate self-reactive TCRs, unlike affinity enhanced TCRs or bispecific antibodies. Therefore, it is hypothesized that the risk of off target reactivity for our TCRs is fairly low, which was confirmed by our safety assessment assays (described below).

Selection of Top MAGEA4 pMHC-Specific TCR-T Cells

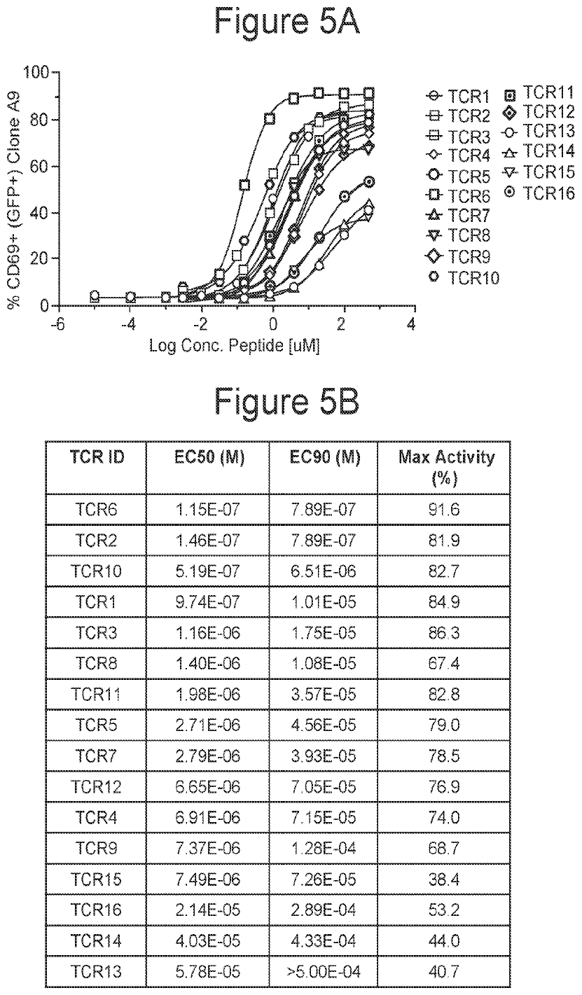

Out of 101 dominant MAGE-A4 pMHC-specific TCRs identified from a screen of 72 healthy HLA-A*02:01+ donors, 20 TCR candidates were selected by a Jurkat activation assay (FIG. 5). Lentivirus carrying individual TCRs were transduced into a Jurkat TCR KO reporter cell line expressing Renilla luciferase that is regulated by TCR activation under a NFAT response element driven promoter. The antigen-specific activity of individual TCR was measured as the fold change of the luciferase activity in the presence of T2 cells loaded with the MAGE-A4 peptide compared to T2 cells with vehicle only (FIG. 5).

### Example 3—Potency Validation of TCR-T-IL12 Cells

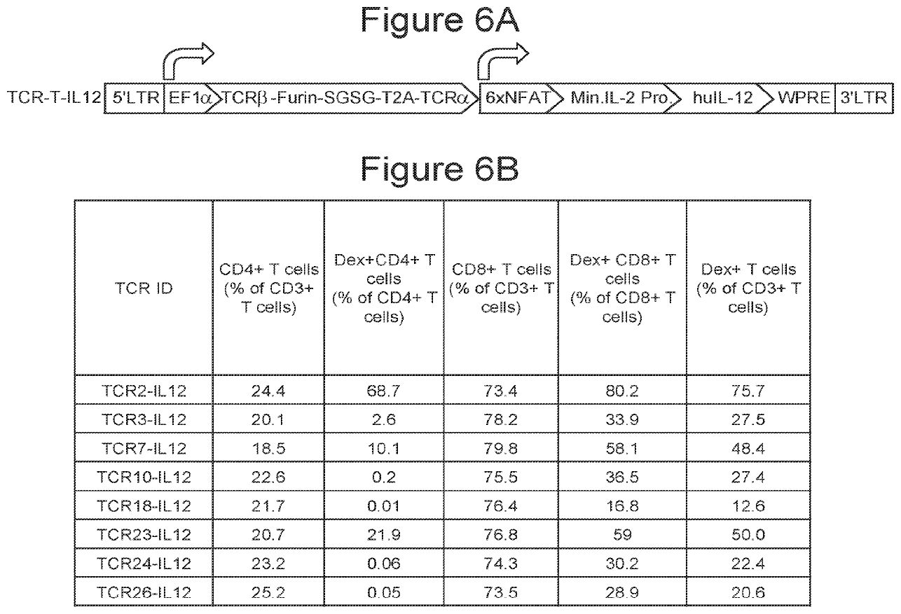

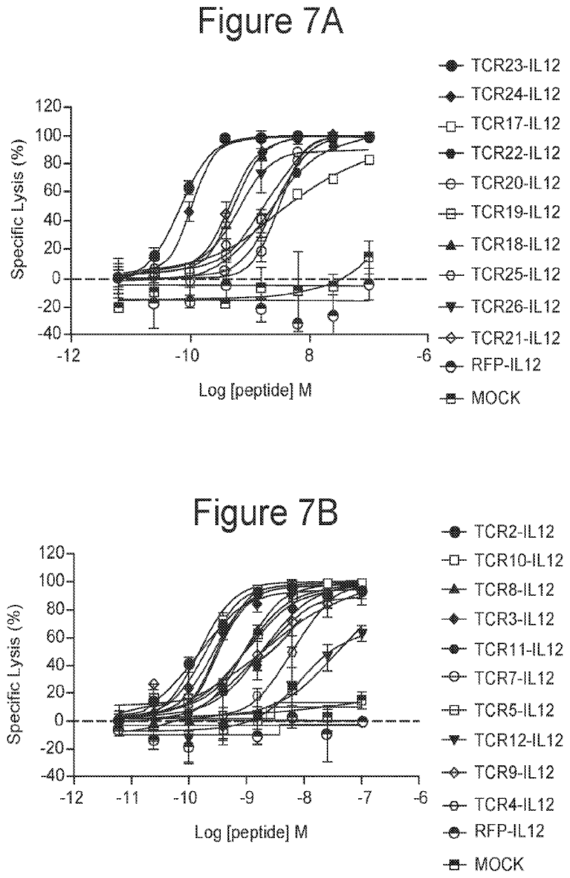

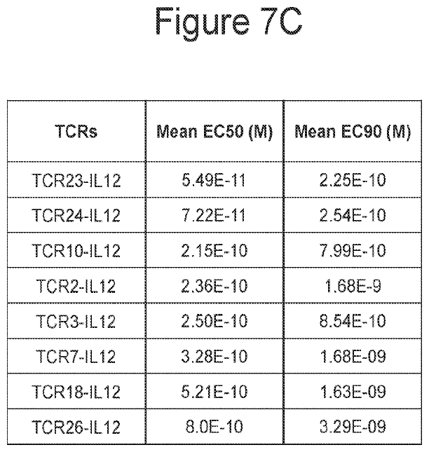

Twenty TCRs (Ten TCRs targeting the MAGEA4 KVLEHVVRV and ten TCRs targeting GVYDGREHTV epitope) were further manufactured in a TCR-T-IL12 lentiviral construct, where the IL12 payload expression is regulated by TCR activation under a NFAT response element driven promoter (FIG. 6A). Therefore, when TCR-T-IL12 cells bind to the pMHC targets in tumors, the IL12 is produced upon TCR signaling, which limits the IL12 secretion predominantly within tumors. Transduction efficiency of TCRs to primary human T cells during TCR-T production was measured by flow cytometry (FIG. 6B). Those TCR-T cells were further evaluated by various functional assays. First, potency of each TCR-T was assessed by using T2/peptide cytotoxicity assays (MAGE-A4 peptide) including peptide titration and E:T (effector:target cell ratio) titration assays (FIG. 7A-C). T2 is a TAP-deficient cell line expressing HLA-A*02:01. As the T2 cell line lacks the transporter for MHC class I-restricted endogenous peptides to enter the ER and presents mainly exogeneous peptides, the T2/peptide cytotoxicity assay (cytolytic activity measurement using T2 cell line loaded by a peptide of interest) were used to study the specific recognition of peptides (e.g. HLA-A*02:01-restricted) by TCR-Ts. The average potency of the top 8 TCRs were identified on the basis of EC50 and presented in FIG. 7C. All of the top eight TCR-IL12 cells met a potency criterion of 10⁻⁸M in EC90 by T2/peptide cytotoxicity assay.

### Example 4—Cross-Reactivity Evaluation of TCR-T IL12 Cells

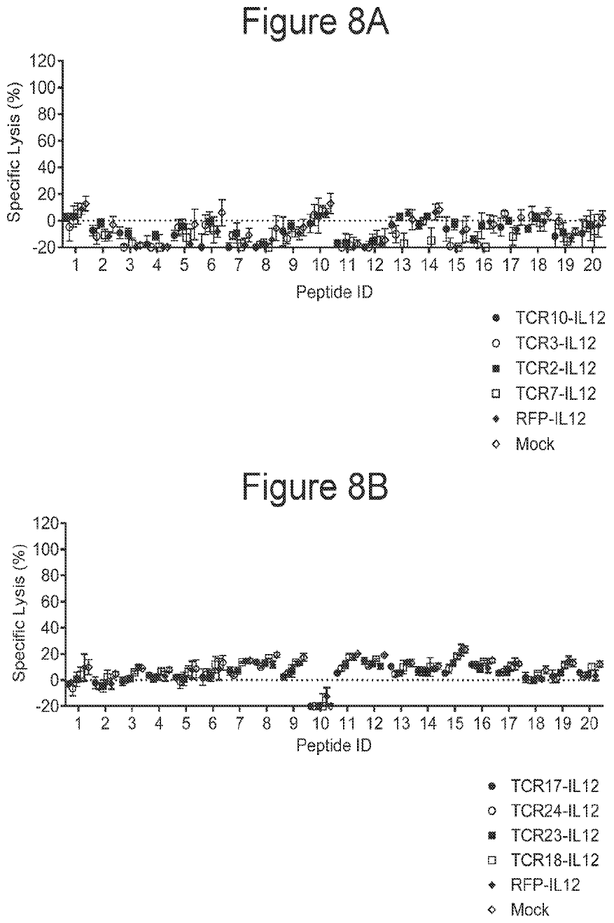

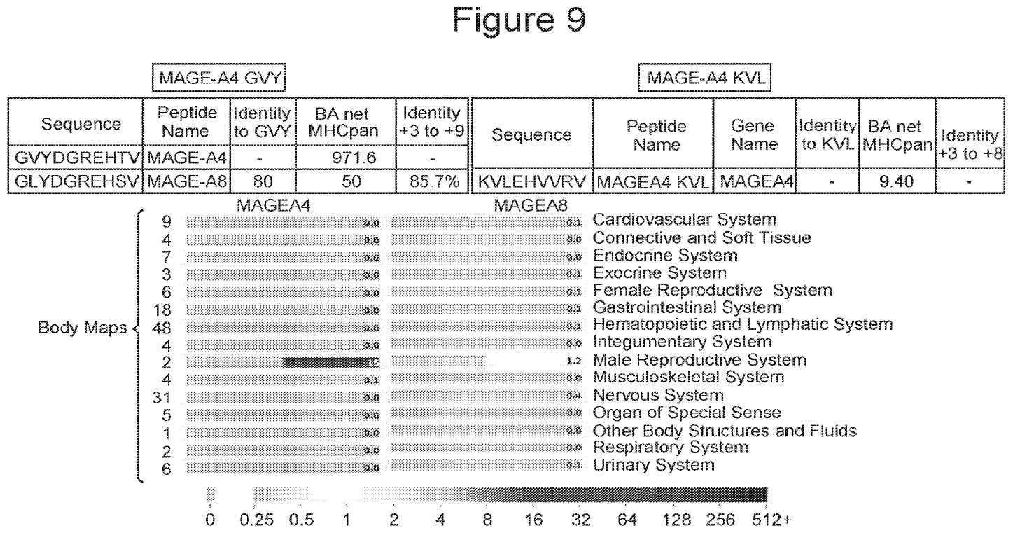

An extensive in vitro and ex vivo safety assessment for TCR-T-IL12 cells as the human-specific HLA target precludes use of animal models. First, for target expression, MAGE-A4 is a cancer testis antigen with extremely restricted normal tissue expression (only expressed in testis). The target expression was assessed by RNASeq, IHC, and mass spectrometry using normal human tissues as well as tumor tissues, which were described above. Second, a critical safety consideration is off-target reactivity, which was evaluated by the T2/peptide cytotoxicity assay using 20 homology-based similar peptides for each TCR against their respective target. No cross-reactivity was observed for any of the top 8 TCRs, potentially supporting the merit of screening naturally occurring TCRs for candidate selection (FIG. 8). The top 20 similar peptides for each of the GVYDGREHTV (SEQ ID NO:1) and KVLEHVVRV (SEQ ID NO:2) epitopes are presented in table 2 and 3, respectively. TCRs were also screened for cross-reactivity against the related CTA MAGEA8. Similar to MAGEA4, MAGEA8 is aberrantly expressed in a variety of tumors, and its expression in healthy tissue is largely restricted to the male reproductive system (FIG. 9).

TABLE 2
Top 20 similar peptides used in cross-reactivity screen for the
GVYDGREHTV MAGEA4 Epitope.

| Peptide | SEQ ID | | Peptide | Identity | BA | Identity +3 |
|---|---|---|---|---|---|---|
| ID | NO: | Sequence | Name | to GVY | netMHCpan | to +9 |
| 1 | 83 | GLYDGPVHEV | DPYSL4 | 60% | 7.8 | 57.1% |
| 2 | 84 | GLYDGPVCEV | DPYSL2 | 50% | 10.2 | 42.9% |
| 3 | 85 | FFVYDEPGHAV | MYOF | 50% | 54.4 | 42.9% |
| 4 | 86 | GVYGGSVHEA | CDNP2 | 50% | 1549.4 | 42.9% |
| 5 | 87 | GVIDGHIYAV | KEAP1 | 50% | 21.3 | 28.6% |
| 6 | 88 | FLSDPQVHTV | DYNC1H1 | 40% | 6.8 | 42.9% |
| 7 | 89 | YTYDEAIHSV | STXBP5 | 40% | 31.8 | 42.9% |
| 8 | 90 | FLLDGFPRTV | AK2 | 40% | 3.4 | 42.9% |
| 9 | 91 | TVYGGYLCSV | COX14 | 40% | 47.5 | 28.6 |
| 10 | 92 | VLFTGREFFV | DDAH | 40% | 8.1 | 42.9% |
| 11 | 93 | DSGDGVTHTV | ACTB | 50% | 25414.6 | 57.1% |
| 12 | 94 | SQYSGQLHEV | OSBPL10 | 40% | 57.6 | 42.9% |
| 13 | 95 | LLKEGEEPTV | PGRMC1 | 40% | 1130.8 | 42.9% |
| 14 | 96 | GLMDGSPHFL | VPS13B | 40% | 8.9 | 42.9% |
| 15 | 97 | VLEDGPWKTV | ATOH8 | 40% | 131.5 | 42.9% |
| 16 | 98 | ALLDGRLQVV | FAS | 40% | 23.9 | 42.9% |
| 17 | 99 | GLFDGVPTTA | PLD4 | 40% | 36.6 | 42.9% |
| 18 | 100 | IIADGGIQTV | IMPDH1 | 40% | 149.5 | 42.9% |
| 19 | 101 | FLYTGEGTV | XIAP | 40% | 32.4 | 42.9% |
| 20 | 102 | NOWDGTQHGV | UTRN | 40% | 306.2 | 42.9% |

TABLE 3
Top 20 similar peptides used in cross-reactivity screen for the KVLEHVVRV
Epitope.

| Peptide | SEQ ID | | Peptide | Identity to | BA | Identity +3 |
|---|---|---|---|---|---|---|
| ID | NO: | Sequence | Name | GVY | netMHCpan | to +9 |
| 1 | 103 | KVLEILHRV | HERC4 | 66.7% | 8.2 | 50.0% |
| 2 | 104 | KVLERVNAV | PSME2 | 66.7% | 19.4 | 50.0% |
| 3 | 105 | KILEDVVGV | TP2 | 66.7% | 5.2 | 66.7% |
| 4 | 106 | KVLETLVTV | HTR5A | 66.7% | 7.7 | 50.0% |
| 5 | 107 | FLLETVVRV | RG1L | 66.7% | 2.6 | 83.3% |
| 6 | 108 | KVLGIVVGV | CNOT1 | 66.7% | 10.5 | 50.0% |
| 7 | 109 | KILEALQRV | ATAD2 | 55.6% | 13.8 | 50.0% |
| 8 | 110 | KLLEQVNRI | GPC5 | 55.6% | 10.7 | 66.7% |
| 9 | 111 | KVLDKVFRA | MIA3 | 55.6% | 62.6 | 50.0% |
| 10 | 112 | KLLDLQVRV | SYNE3 | 55.6% | 4.8 | 50.0% |
| 11 | 113 | IVMEHVVFL | ANO5 | 55.6% | 5.8 | 66.7% |
| 12 | 114 | ILDEHVORV | AXIN1 | 55.6% | 4.9 | 66.7% |
| 13 | 115 | KVTHAVVTV | GRP78 | 55.6% | 177.7 | 33.3% |
| 14 | 116 | KLLEKVRKV | PTRF | 55.6 | 21.1 | 50.0% |
| 15 | 117 | VALEHFVLV | TRMT1L | 55.6% | 159.3 | 66.7% |
| 16 | 118 | KVLNKVITV | KIAA1033 | 55.6% | 26.7 | 33.3% |
| 17 | 119 | KVQEQVHKV | NSD1 | 55.6% | 194.7 | 33.3% |
| 18 | 120 | KVWGNVVTV | HNRNPR | 55.6% | 10.0 | 33.3% |
| 19 | 121 | SLLGHVIRL | TSC1 | 44.4% | 8.0 | 66.7% |
| 20 | 122 | ILLEHKVVL | MADD | 44.4% | 91.23 | 66.7% |

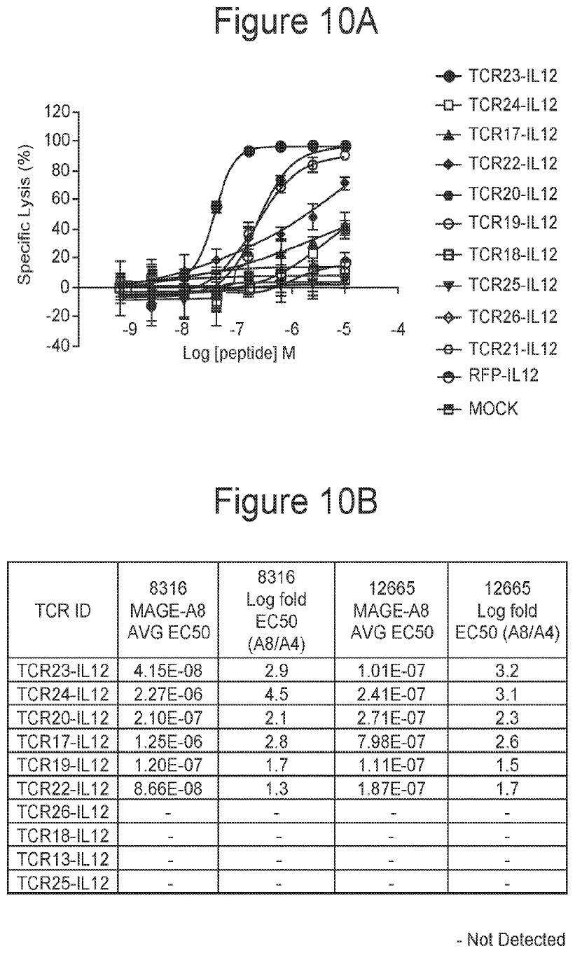

MAGEA4 and MAGEA8 share significant sequence homology. Notably, in the region of the target GVYDGREHTV (SEQ ID NO:1) peptide targeted by TCR-Ts in this work, the corresponding MAGEA8 peptide (GLYDGREHSV (SEQ ID NO:123) shows 80% sequence identity. The KVLEHVVRV (SEQ ID NO:2) peptide is 100% identical between both MAGEA4 and MAGEA8 proteins and would therefore provide no basis for differential activity in cognate TCR-Ts. GVYDGREHTV-MHC cognate TCRs were screened for differential activity against the two peptide epitopes, revealing a greater than ˜1000-fold difference in reactivity of previously identified top TCRs (FIG. 10). These data demonstrate that the top 4 GVYDGREHTV-MHC cognate TCRs identified here may be used to specifically target tumors with little risk of MAGEA8 cross-reactivity. By contrast, the top KVLEHVVRV-MHC cognate TCRs will have likely utility in killing a broad set of cancerous cells with expression of MAGEA4, MAGEA8, or both.

### Example 5—Cytotoxicity Against MAGEA4+ Cancer Cell Lines

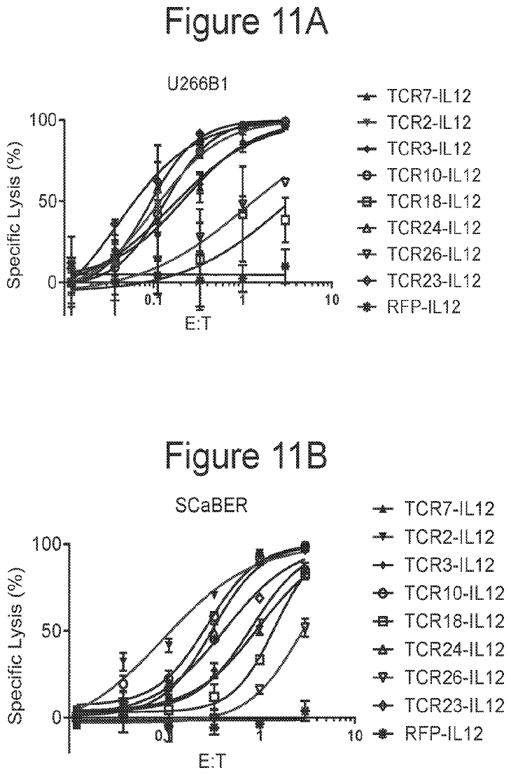

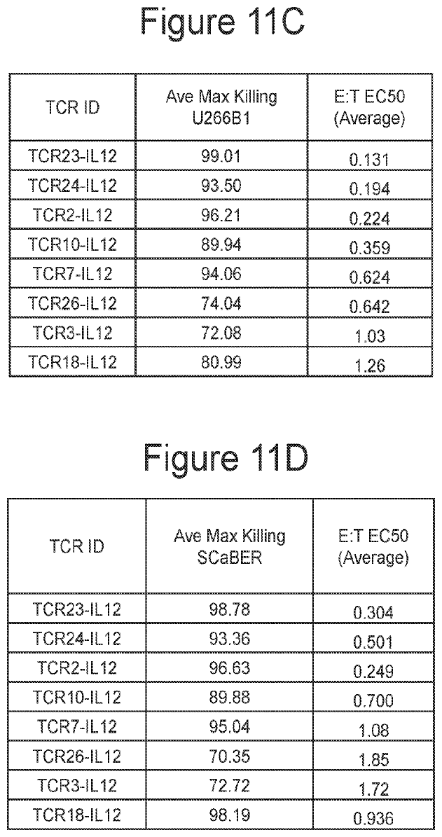

Top 8 TCRs were evaluated in TDCC assays against cancer cell lines. All 8 TCRs demonstrated cytotoxicity against MAGEA4+ cell lines U266B1 and SCaBER (FIGS. 11A and B). Measured EC50 against both cell lines identified a consistent set of top 5 TCRs which were selected and progressed to subsequent studies. Notably, the top 5 TCR-Ts expressed TCRs in greater than 20% of CD8+ T cell following lentiviral transduction. In conclusion, these TCRs were selected based on (1) potent cytotoxicity MAGE-A4 pMHC targets evaluated through TDCC against T2/MAGEA4 peptide and MAGE-A4+ cancer cell lines, (2) off-target selectivity showing no cross-reactivity against 20 homology-based similar peptides and target negative cancer cell lines, and (3) manufacturability (e.g. good TCR transduction efficiency).

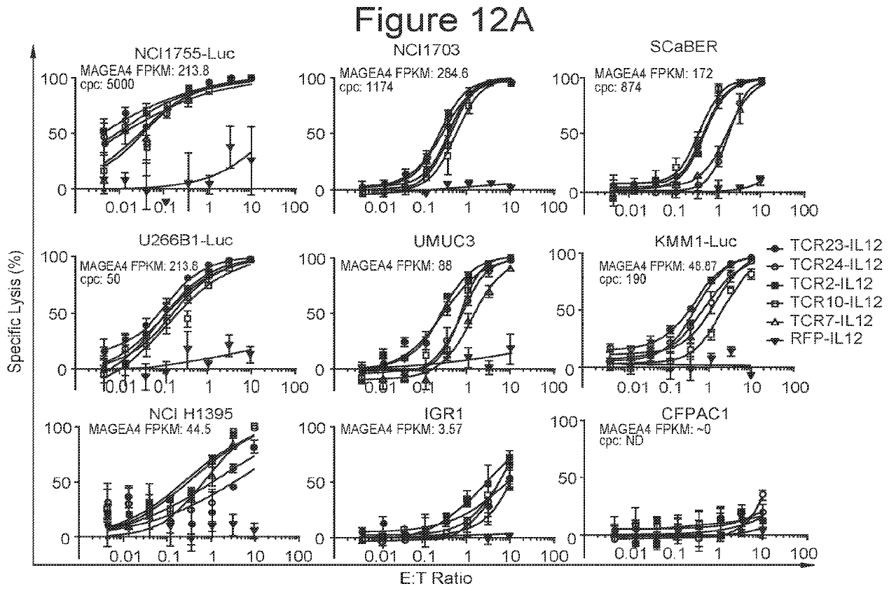

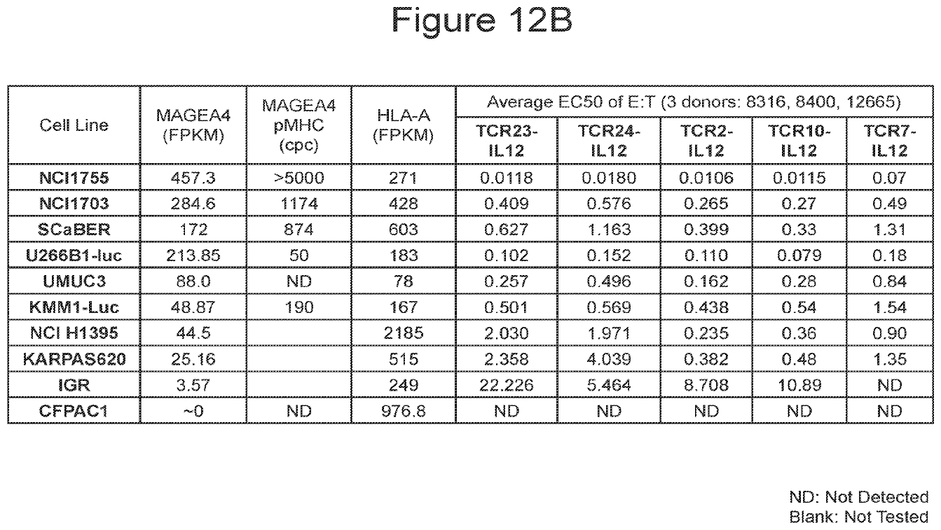

The potency (cytotoxicity) of the top five TCR-T-IL12 were validated using a larger set of HLA*0201+ cancer cell lines spanning a range of MAGEA4 expression (FIG. 12A). All 5 TCR-T-IL12s displayed potent cytotoxicity against cancer cell lines with MAGE-A4 expression as low as ˜3.6 FPKM. All four TCRs were similarly potent, making relative ranking of top TCRs challenging within one cell line. Using an aggregate EC50 ranking system against MAGEA4+ cells however, TCR2 and TCR10 were the most potent, followed by TCR23, TCR24, AND TCR7.

Representative cancer cell line potency data of the top five TCR-T-IL12 cells are shown in FIG. 12B. 10 cancer cell lines were tested with four TCR-T-IL12 cells generated from 2-3 donors. TCR-T-IL12 cells demonstrated potent cytotoxicity against some cancer cell lines with low E:T EC50. For example, E:T EC50 of TCR2 TCR-Ts against NCI-H1755 (FPKM=457.3) was on average 0.01 across experiments using TCR-Ts generated from three different donors, indicating that TCR-Ts were able to display significant cytotoxicity against tumor cells even when outnumbered by 100:1.

### Example 6—Overview of Nonclinical Safety Assessment

An extensive in vitro and ex vivo safety assessment for TCR-T-IL12 cells was performed, as the human-specific HLA target precludes the use of animal models. First, the target expression was assessed by various assays including RNASeq, IHC, and mass spectrometry using normal human tissues as well as tumor tissues, which were described above. As MAGE-A4 is a cancer testis antigen, our studies displayed extremely restricted normal tissue expression (only expressed in testis). Second, off-target reactivity was assessed using two different strategies. The first strategy involved screening to evaluate cytotoxicity against various normal human primary cells. The second strategy involved identifying a panel of similar peptides based on sequence homology match to the MAGE-A4 target peptide along with a positional scanning (X-Scan motif)-based strategy to identify putative cross-reactive peptides unique to each TCR. To access potential cross-reactivity to this full panel of similar peptides, T2/peptide TDCC assays were conducted. The third safety assessment was alloreactivity, which was assessed using 34 BLCLs (B lymphoblastoid cell lines) representing highly frequent HLA class I alleles in US populations, including 38 HLA-A, 40 HLA-B, and 24 HLA-C alleles.

### Identification of Similar Peptides Based on Homology

To assess off-target reactivity an in silico approach was used to identify a list of peptides with high sequence similarity that could potentially cross-react with the candidate TCR-Ts. To accomplish this a protein database restricted to Homo sapiens (UniProtKB/Swiss-Prot, June 2019) was first queried using a Python script to generate a list of all possible peptides based on sequence identity to the target. This in silico query using GVYDGREHTV was performed using a Python script and resulted in the identification of 137,999 decameric peptides based on a 30% homology (identity) match to the target peptide. To further refine this list criteria such as high homology match, and software such as NetMHCpan and IEDB (The Immune Epitope Database) were utilized. NetMHCpan3.0 software was used to consider a peptide's predicted binding affinity to HLA-A*02:01. IEDB database (June 2019), which is a manually curated database of experimentally characterized immune epitopes, was used to consider a peptide's chance of being processed and presented by the HLA-A*02:01 allele. Specific criteria used for peptide selection were as follows, (1) all HLA-A*02:01+ peptides in IEDB with ≥40% homology match (identity) to the target peptide (40 peptides), (2) all peptides with ≥60% homology match and predicted HLA-A*02:01 binding affinity (IC50)<5000 nM (51 peptides), and (3) all peptides with ≥50% homology match to target peptide that are reported in IEDB (presented by HLA-A*02:01 allele) (53 peptides). As a result, this homology-based in silico search of the human proteome database and filtering criteria led to the identification of 144 unique peptides for screening of GVYDGREHTV TCR-23 and TCR-24.

For the MAGEA4 target peptide KVLEHVVRV the same in silico search resulted in the identification of 155,353 nonameric peptides based on a 30% homology match to the target peptide. To further refine the list of peptides the following criteria were used, (1) all HLA-A*02:01+ peptides in IEDB with ≥40% homology match (identity) to the target peptide (176 peptides), and (2) all peptides with ≥60% homology match and predicted HLA-A*02:01 binding affinity (IC50)<5000 nM (50 peptides). As a result, this homology-based in silico search of the human proteome database and filtering criteria led to the identification of 226 unique peptides for screening of KVLEHVVRV TCR2 and TCR10.

### Identification of the TCR Binding Motif Using Positional Scanning (X-Scan) and Similar Peptides Based on X-Scan-Derived Motifs

As an orthogonal approach to identify similar peptides, we used a positional scanning method, known as X-scan. This assay uses a peptide library that is generated by sequentially mutating each residue of the MAGE-A4 peptides to one of the other 19 naturally occurring amino acids, resulting in a total of 171 peptides for the KVLEHVVRV target and 190 peptides for the GVYDGREHTV target. These peptides were synthesized and tested in the T2/peptide TDCC assay to identify an X-scan derived motif that is specific to each individual TCR (Table 3). Briefly, T2 cells were pulsed with each of these peptides at a 10 μM concentration, followed by addition of TCR-T cells at an E:T ratio of 1:1. Cell viability was determined using a T2/peptide TDCC assay. An amino acid substitution was defined as essential for TCR2/TCR10 engagement where the viability observed was less than 30% and less than 40% for TCR23/TCR24. A corresponding search motif was constructed to express which amino acids were tolerated at each position in the peptide sequence (Table 3). Underlined amino acids represent the native residue at the corresponding position in the peptide. Utilizing the motifs generated an in silico search of the human proteome (Homo sapiens restricted UniProtKB/Swiss-Prot database with splice variants June 2019) was performed to identify all decameric (TCR23/TCR24) or nonameric (TCR2/TCR10) sequences that comply with the derived motif. This approach resulted in an additional set of peptides for off target screening that are specific to individual TCR-Ts (Table 3). To determine if these sequences would lead to off-target toxicity if presented on the cell surface a panel of peptides for TCR2 (21) and TCR23 (1) were synthesized and cell viability was tested using a single point T2/peptide (10 μM) TDCC assay as described above. All three donors showed greater than 70% cell viability against the full panel of peptides indicating no off-target liabilities from either TCR2 or TCR23.

TABLE 3

| | | Unique | |
|---|---|---|---|
| | | peptide | |
| | | matches, | |
| | | which | |
| | | conform | |
| | | to the | SEQ |
| | Motif obtained through | consensus | ID |
| TCR | Positional Scanning | motif | NO: |
| TCR2 | [KCMR][VDTSEACMILN][LTPG | 21 | 123 |
| | ACVMIYFKWQ][E][HCYF][VDT | | |
| | SEPGACMILYFHKRWQN][VI][R | | |
| | E][VTSGAC] | | |
| TCR10 | [KVIR][VTSCILY][LSPGACVM | 18 | 124 |
| | IYFHRWQN][E][HTF][VSGACM | | |
| | ILY][VCIFW][R][VTSEPGACM | | |
| | ILFQ] | | |
| TCR23 | [GDTSAIYFN][VI][Y][D][GM | 1 | 125 |
| | ILYFHRWQN][RPIK][ECR][HP | | |
| | YW][TDSEPGACVMILYFHKQN] | | |
| | [VCILF] | | |
| TCR24 | [GDTSEPACVMILYFHKRWQN][V | 33 | 126 |
| | TSAMILYQ][YFW][DMN][GTSP | | |
| | ACVMILYFHKRWQN][RPIK][ED | | |
| | CMLYFHQN][H][TSPGACVMILH | | |
| | WQN][VTACMILF] | | |

Cross-Reactivity Screen with Full Panel Similar Peptides

Full panel similar peptides were synthesized and examined in T2/peptide TDCC assays to investigate the likelihood of off-target reactivity. To identify potential cross-reactive peptides for each TCR-T-IL12, the full panel of similar peptides was tested using a T2/peptide TDCC screen with a high peptide concentration (10 μM). Peptides that showed less than or equal to 25% viability in at least one of three donors were considered as putative cross-reactive peptides and were selected for a further potency test.

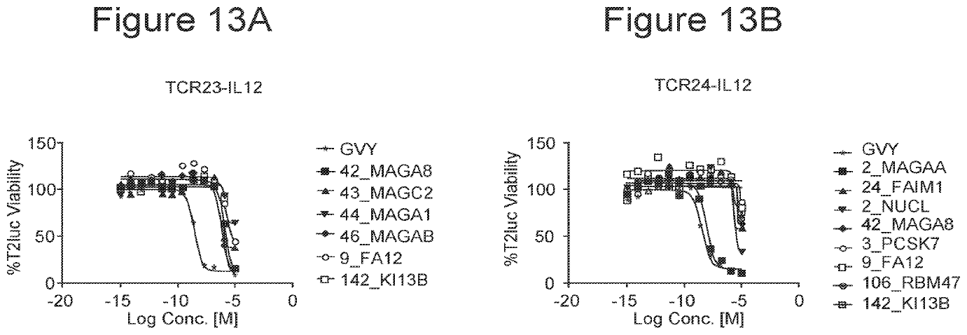

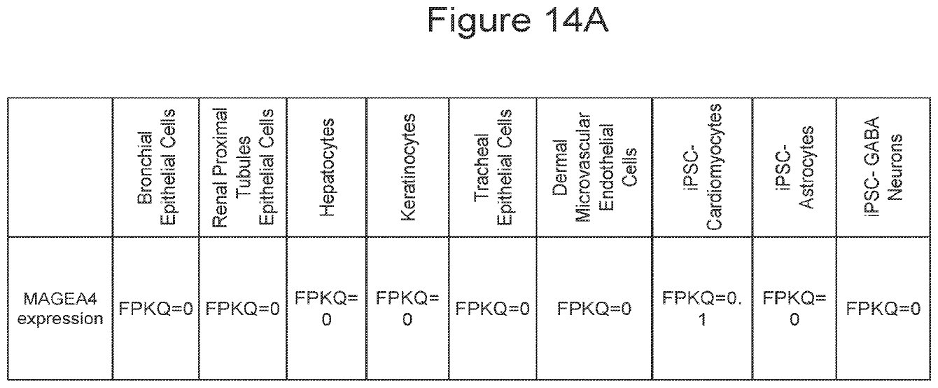

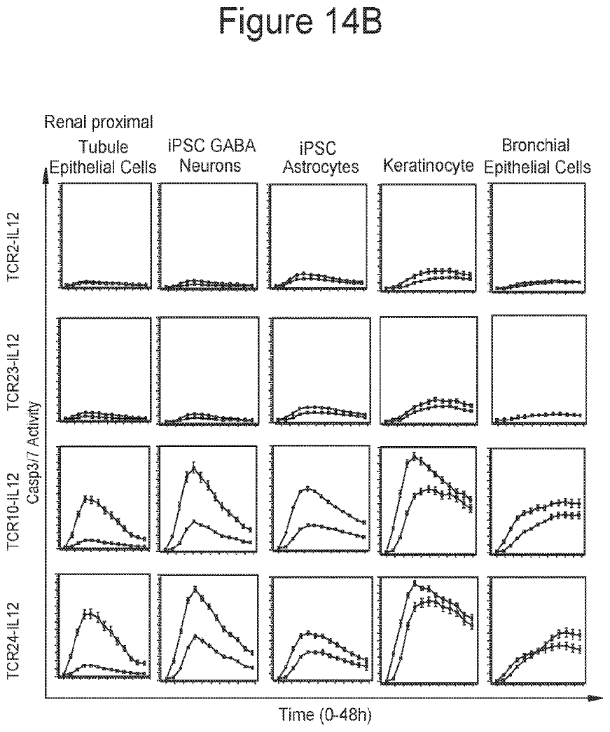

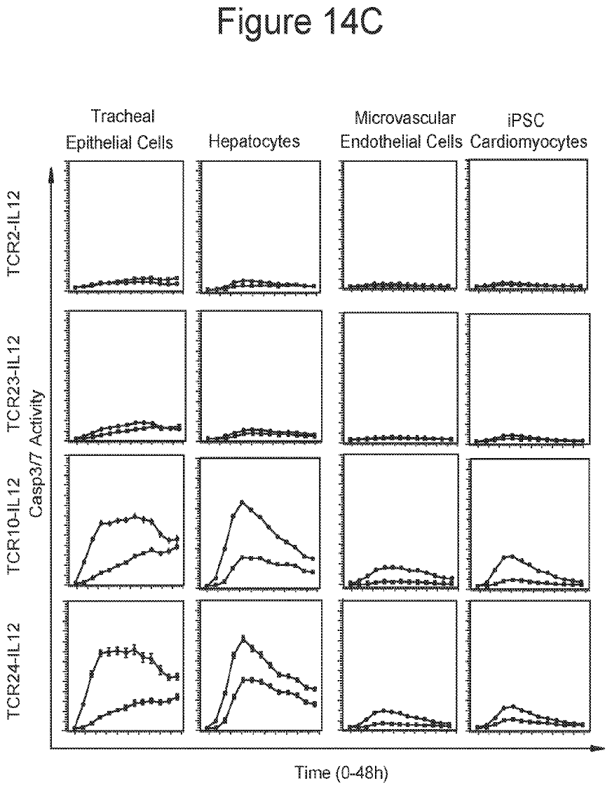

Next, a potency screen (dose-dependent screen) was performed using T2/peptide titration TDCC assays for the putative cross-reactive peptides (Table 4) identified from the above screen (FIG. 13A-B). A potency gap of less than 10³-fold in EC50 between target peptide and putative cross-reactive peptides was considered as a cutoff for future risk assessment. This methodology yielded no putative cross-reactive peptides for TCR2 and TCR10, while multiple putative cross-reactive proteins were identified for both TCR23 (6 peptides) and TCR24 (8 peptides). Likely due to high sequence homology within the MAGE protein family, both TCR23 and TCR24 were found to have cross-reactivity to multiple type 1 MAGE family proteins such as MAGEA8, MAGEA10, and MAGEA11. Although these findings are worthy of further investigation, due to their membership in cancer testis antigen class, these putative cross-reactivity against these MAGE family derived peptides were not considered to be a significant safety risk. However, these assays also identified two potential non-MAGE family proteins as potential cross-reactive liabilities: FA12 and KI13B for TCR23 and NUCL and RBM47 for TCR24. Given the large potency gap (>100 fold) and lack of experimentally verified endogenous HLA-A*02:01 presentation of these cross-reactive similar peptides, the relevance of these cross-reactivities as they relate to the clinical liability of TCR23 and TCR24 is not yet clear. In conclusion, all TCRs were found to be unreactive against the vast majority of proteome derived similar peptides. No liabilities were found for TCR2 and TCR10, while two putative cross-reactive peptides derived from non-CTA proteins were identified for TCR23 and TCR24. It is noteworthy that TCR2 and TCR23 exhibited minimal caspase 3/7 cleavage when co-cultured with any of the human normal primary cells or iPSC-derived cells tested, indicating no obvious off-target reactivity against any of the normal cells tested, which can present highly diverse peptides (see section below; FIG. 14 B, C).

TABLE 4
Peptides identified from the similar peptide screen

| | | | | SEQ | |
|---|---|---|---|---|---|
| | | | | ID | % |
| Accession | Protein | Gene | Sequence | NO: | Identity |
| P43361 | MAGA8 | MAGEA8 | GLYDGREHSV | 127 | 80% |
| Q9UBF1 | MAGC2 | MAGEC2 | GVYAGREHFV | 128 | 80% |
| P43355 | MAGA1 | MAGEA1 | EVYDGREHSA | 129 | 70% |
| P43364 | MAGAB | MAGEA11 | GVYAGREHFL | 130 | 70% |
| P43363 | MAGAA | MAGEA10 | GLYDGMEHLI | 131 | 60% |
| Q16549 | PCSK7 | PCSK7 | VVDDGVEHTI | 132 | 60% |
| P00748 | FA12 | F12 | YLAWIREHTV | 133 | 50% |
| A0AV96 | RBM47 | RBM47 | MIEDGKIHTV | 134 | 50% |
| Q9NQT8 | KI13B | KIF13B | LVYYLKEHTL | 135 | 50% |
| Q9NVQ4 | FAIM1 | FAIM | FVDDGTETHF | 136 | 40% |
| P19338 | NUCL | NCL | GEIDGNKVTL | 137 | 40% |

### Assessment of Cytotoxicity Against Human Primary Normal Cells

Next, cytotoxicity of TCR-T-IL12 cells against various human primary normal cell types representative of vital organs (with no MAGE-A4 expression) serving as target cells was evaluated in a T-cell mediated cytotoxicity assay. TCR2, TCR10, TCR23, and TCR24 were tested against a panel of human normal primary cells or iPSC-derived cell lines representative of vital organs, including bronchial epithelial cells (hBEpC), tracheal epithelial cells (hTEpC), dermal microvascular endothelial cells (HDMEC), keratinocytes, hepatocytes, renal proximal tubule epithelial cells (RPTEC), iPSC-derived astrocytes, cardiomyocytes, and GABA neurons (FIG. 14A). All primary cells and iPSC-derived cell lines were derived from the normal tissues of HLA-A*02:01-positive donors. Importantly, as those normal cells can present highly diverse peptides on HLA-A*02:01, this serves as an assay system to assess a broad range of off-target effects. Mock (untransduced) T cells or T cells expressing an IL12-RFP construct (with no transgenic TCR) from the same donor were included as negative control effector cells. Nine normal primary cell types were assessed for cytotoxicity measured by caspase 3/7 cleavage assay in the presence of TCR-T cells or NFAT.IL12 T cell controls. Importantly, TCR2 and TCR23 exhibited minimal caspase 3/7 cleavage when co-cultured with any of the normal primary cells or iPSC-derived cells tested when compared to the IL12 T cell control, indicating no obvious off-target reactivity against any of the non-cancerous cells tested (FIG. 14 B, C). By contrast, measured caspase 3/7 response was dramatically higher than control T cells for TCR10 and TCR24 TCR-Ts across all tested non-cancerous cells (FIG. 14 B, C).

### Assessment of Alloreactivity Using 34 BLCL Lines

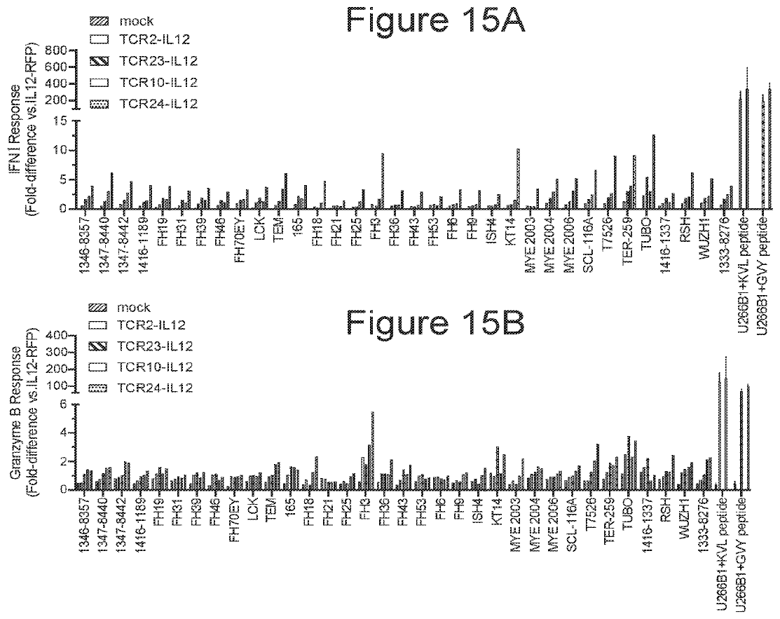

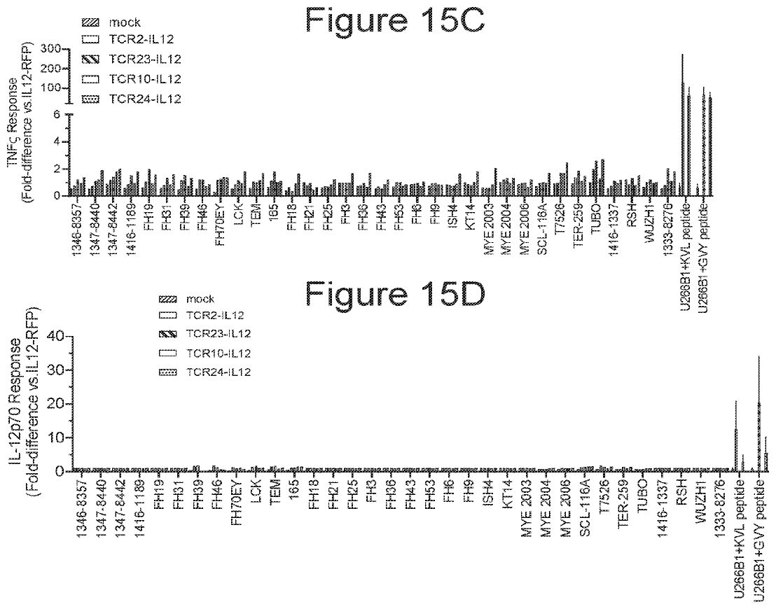

As a part of safety assessment, alloreactivity potential was evaluated by using 34 BLCL lines (B lymphoblastoid cell lines) representing highly frequent (≥11%) MHC Class I alleles in major US ethnic groups, including 39 HLA-A, 40 HLA-B, and 23 HLA-C alleles. Alloreactivity potential was evaluated by the production of cytokines (IFNγ, TNFα, and IL-12p70) and granzyme B. No significant increases in cytokine or granzyme B responses against the 34 BLCL lines tested were observed for TCR2 transduced T cells (FIG. 15A-D). Only low-level threshold responses were observed for TCR23 T cells in 3/34 BLCLs, defined as greater than or equal to 3-fold induction in any one analyte compared to IL12-RFP control cells (FIG. 15A-D). By contrast, larger alloreactive responses were observed for TCR10 and TCR24, with a clear induced cytokine and granzyme B response detected in 31/34 BLCLs for TCR24. All four TCR-T-IL12 cells demonstrated robust cytokine and granzyme B responses against a positive control U266B1 cells (HLA-A*02:01+ MAGE-A4+) pulsed with MAGEA4 peptide (GVYDGREHTV or KVLEHVVRV) (FIG. 15A-D).

Overall, these studies identify exemplary TCR candidates that do not show significant safety concerns based on the normal and alloreactivity potential safety assessments performed.

### Example 7. TCRs Demonstrate Potent Cytotoxic Activity Against Homologous HLA-A pMHC

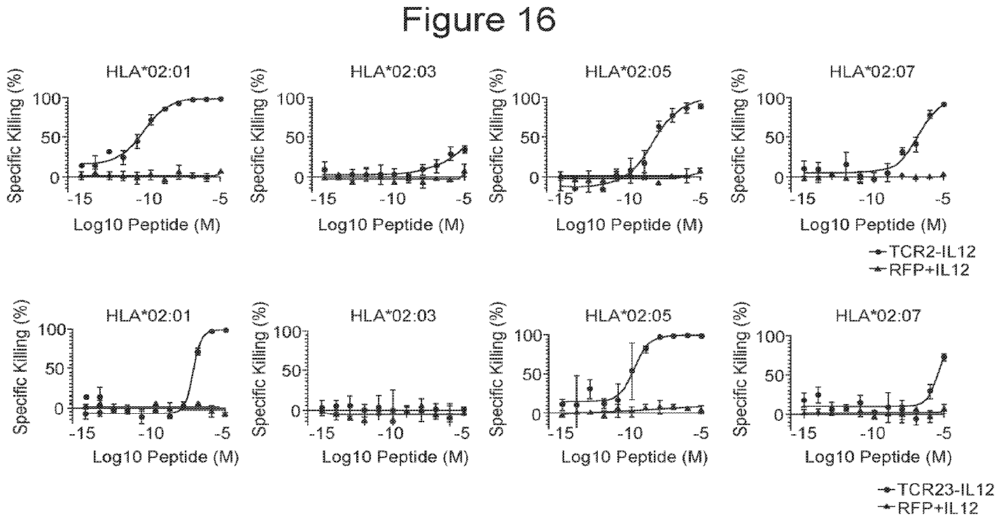

TCR2 and TCR23 TCR-Ts were assessed for potency in a using peptide loaded CIR cells expressing mismatched HLA-A alleles bearing high sequence identity to HLA-A*02:01 (FIG. 16). To generate these allelic variants of CIR, an HLA-A deficient cell line, CIR cells were transduced with a to express HLA-A*02:01, HLA-A*02:03, HLA-A*02:05, HLA-A*02:06, or HLA-A*02:07. These transduced CIR cells expressing new HLA-A2 alleles were peptide pulsed with KVLEHVVRV or GVYDGREHTV peptide before use as target cells in TDCC assays. Both TCR2 and TCR23 demonstrated cytotoxic activity against MAGEA4 peptide loaded CIR cells expressing HLA-A*02:01 and HLA-A*02:05, and additionally TCR23 for HLA-A*02:06, and TCR2 for HLA-A*02:07. These studies suggest that the utility of TCRs may be not only limited to HLA-A*02:01+ patients.

### Methods and Materials Used in the Above Examples

MAGE-A4 pMHC-Specific TCR Identification by Healthy Donor Screen

### Generation of Autologous Antigen Presenting Cells (APCs)

HLA-A*02:01 positive healthy donor peripheral blood mononuclear cells (PBMCs) were obtained from leukopak from AllCells or PPA. Monocytes were positively selected by using human CD14-microbeads (Miltenyi Biotec, 130-050-201) from PBMCs. Mature dendritic cells were obtained by using CellXVivo Human Monocyte-derived Dendritic Cell (DC) Differentiation Kit (R&D, CDK004). Antigen presenting B cells were generated by using CD40L and IL-4 stimulation method. B cells were positively selected by using human CD19-microbeads (Miltenyi Biotec, 130-050-301) from PBMCs. CD19+ cells were then stimulated by 0.125 ug/ml recombinant huCD40L in B cell media and seeded in 24-well plate at 2×10⁵ cells/ml and 1 ml/well. B-cell media comprised of IMDM, GlutaMax supplement media (Gibco, 31980030) supplemented with 10% human serum (MilliporeSigma H3667-100 ML), 100 U/ml penicillin and 100 μg/ml streptomycin (Gibco, 15140-122), 10 μg/ml gentamicin (Gibco, 15750-060) and 200 IU/ml IL-4 (Peprotech, 20004100UG). Fresh B cell media with 400 IU/ml IL-4 was added to the B cell culture at 1 ml/well on day 3 post B cell activation without disturbing the cells. Activated B cells were ready to use for antigen-reactive T cell stimulation on day 5 post B cell activation.

### Ex Vivo Stimulation and Expansion of Antigen-Specific T Cells

MAGE-A4 peptide (Anaspec customized peptide) was added to the immature dendritic cells at 1 μM along with recombinant human TNF-α on day 7 post CD14+ cell isolation. On day 9 post CD14+ cell isolation, MAGE-A4 peptide-pulsed mature dendritic cells were collected, washed and mixed with CD14-PBMCs at ratio 1 to 10 in human T cell media with 10 μM MAGE-A4 peptide, 10 IU/ml IL-2 (Miltenyi Biotec, 130-097-745) and 10 ng/ml IL-7 (Peprotech, AF20007100UG). Human T cell complete media consists of a 1 to 1 mixture of CM and AIM-V (Thermo Fisher, 12055083). CM consists of RPMI 1640 supplemented with GlutaMAX (Gibco, 61870-036), 10% human serum (MilliporeSigma, H3667), 25 mM HEPES (Gibco, 15630-080) and 10 μg/ml gentamicin (Gibco, 15750-060). MAGE-A4 specific T cells were further expanded by one to two rounds of weekly peptide pulsed B cell activation. HuCD40L activated B cells were collected, washed and seeded in 6-well plate at 1×10⁶ cells/ml and 4 ml/well, 1 μM MAGE-A4 peptide was added to the B cells and incubated at 37° C. for 2 hours in the incubator. The peptide pulsed B cells were then mixed with the T cells at ratio 1:10 in human T cell media with 10 IU/ml IL-2 and 10 ng/ml IL-7. MAGE-A4 dextramer positive cells were confirmed by flow cytometry and then sorted for TCR identification by single cell RNAseq.

### Sorting of Activated Antigen-Specific T Cells

MAGE-A4 peptide activated antigen specific T cells were stained with MAGE-A4 dextramer-APC and -PE at room temperature in dark for 10 min and then stained by CD3-FITC (Biolengend, 300440) and CD8-BV605 (BD Biosciences, 564116). The dead cell exclusion stain (Sytox blue) was purchased from ThermoFisher (Invitrogen, S34857). Cells were sorted using an Aria Fusion cell sorter (BD Biosciences, San Jose, CA). Data were analyzed using Flowjo post-sort.

### ELISPOT

The sorted CD3+CD8+Dex+ T cells were validated for the antigen-specific IFNγ production by ELISPOT assay (BD, 551849) using peptide-loaded T2 cells. T2 cells were loaded with 10 μM MAGE-A4 peptide in human T cell complete media at 2×10⁶ cells/ml and 1 ml/well in 24 well plate for 1-2 hours. 150 ul of human T cell complete media and 50 μl of peptide loaded T2 cells were added to each well in the pre-coated ELISPOT plate. The CD3+CD8+Dex+ T cells (500 or 1000 cells) were directly sorted into each well in the ELISPOT plate. The ELISPOT was detected after 24-hour incubation in 37° C. incubator. The ELISPOT plates were scanned and counted by ImmunoSpot (Cellular Technology Limited, Cleveland, OH).

### Single Cell RNAseq

Samples were processed using a Chromium Controller (10× Genomics, Pleasanton, CA) with the V (D) J single-cell Human T Cell enrichment kit (PN-1000006, PN-1000005, PN-120236, PN-120262) according to manufacturer's instructions for direct target enrichment, skipping cDNA amplification step for the full transcriptome. Briefly, cells and beads with barcoded oligonucleotides were encapsulated in nanoliter droplets where the cells were lysed, and mRNA reverse transcribed with poly-T primers and barcoded template-switch oligos. Nested PCR was then performed with primers in the constant region of the human TCR and template-switch oligo. The second target enrichment PCR was performed using 13-17 cycles depending on estimated cell input number according to manufacturer's suggestions. The final sequencing library was generated from fragmented PCR product ligated to Illumina sequencing adapters. Libraries were sequenced with 151 paired end reads (151×8×0×151) on NextSeq 550 or MiSeq (Illumina, Inc., San Diego, CA) at a depth of at least 5,000 reads per cell. Data was demultiplexed and analyzed with cellranger vdj (2.2.0) to obtain full-length paired TCR sequences assigned to individual cells.

Cloning and Transduction of TCRs into Jurkat Cells

Candidate TCRs were generated as gene fragments. Each fragment was cloned into a plasmid expression vector consisting of a MSCV promoter and an IRES-driven eGFP for monitoring transfection or transduction. Successful transformants were screened by Sanger sequencing and verified clones were maxi-prepped for downstream applications. TCRs were transfected into a Jurkat TCR KO reporter cell line expressing Renilla luciferase under a NFAT inducible promoter. Briefly, 1.5 μg of plasmid was added to 3E5 cells in suspended in 10 μL buffer R (Thermo Fisher Scientific). Cells were electroporated using the Neon™ transfection system according to manufacturer conditions, using a pulse voltage of 1350V, a pulse width of 20 ms, and a pulse number of 2. The contents of the electroporation reaction were then diluted into 200 μL of RPMI 1640 supplemented with 15% heat-inactivated FBS, glutaMAX™, penicillin/streptomycin, and 4.5 g/L D-glucose in a 96 well plate for overnight culture in a 37° C. incubator.

### Jurkat Activation Assay

Antigen-presenting T2 cells (ATCC) were loaded with peptides (Anaspec customized) or vehicle only at a range of concentrations in serum-free media for two hours. After incubation, loaded T2 cells were washed three times before being resuspended in complete media and cell counting. 2E5 peptide loaded T2 cells were then added directly to TCR transfected Jurkat cells directly in 100 μL of complete media. The TCR-expressing Jurkat cells were co-cultured at 37 C in the presence of the T2 cells for 24 hours. Following incubation, cells were transferred to a 96-well U-bottom plate and 150 ul FACS buffer (PBS w/o CaCl₂ & MgCl₂ (Corning, 21-040-CV)+5% FBS (Gibco, 10082-147)) added before being centrifuged at 400×g for 4 min. Supernatant was removed and cells resuspended in 50 μl of 1×Fc block in FACS buffer which was incubated at 4 C for 20 min. αCD69-BV421 or IgG isotype-BV421 was added at a concentration of 1 μg/mL and incubated at 4° C. for 1 hr. Cells were washed three times after staining by centrifugation at 400×g for 4 min followed by aspiration and resuspension. Prior to analysis, cells were suspended in FACS buffer containing Sytox Red prepared according to manufacturer recommendations. Cells were analyzed using either LSRII or Symphony cytometers (BD Biosciences) using recommended acquisition settings. Activity of individual TCRs is presented as the percentage of cells expressing CD69 within the population of GFP expressing Jurkat cells (signifying plasmid expression).

### MAGE-A4 TCR-T Cell Production Using Human Primary T Cells

PBMCs from three healthy donors (HLA-A*02:01) were isolated from leukopak (Allcells) using Ficoll-Paque gradient centrifugation, with additional T cell isolation by using CD3 negative selection kit (Miltenyi Biotec, 130-096-535) and associated manufacturer's protocol. One day before TCR transduction, frozen pan-T cells were thawed and resuspended in Human T cell complete media at 1×10⁶ cells/ml, and were stimulated by CD3/CD28 dynabeads (Thermo Fisher, 11131D) with T cells to beads ratio (2:1) in the presence of 30 IU/ml IL-2 (Miltenyi Biotec, 130-097-745), 10 ng/ml IL-7 (Peprotech, AF20007100UG) and 25 ng/ml IL-15 (Peprotech, AF20015100UG). The T cells were then seeded at 1 ml per well in 24-well plates. On the day of TCR transduction, activated T cells (3E5) were seeded in Human T cell complete media per well in 48-well plate and transduced with lentivirus in the presence of 8 μg/ml polybrene, 100 IU/ml IL-2, 10 ng/ml IL-7 and 25 ng/ml IL-15. The T cells were then spin-inoculated for 1.5 hours at 32° C. After spin-inoculation, 380 ul of media with 8 μg/ml polybrene, 100 IU/ml IL-2, 10 ng/ml IL-7 and 25 ng/ml IL-15 was added to the cells to make total volume 600 μl per well. At 17-18 hours post transduction, ˜500 μl of media was removed without touching the cells at the bottom of the wells. The cells from each well of 48-well plate were transferred to one well of Grex 24-well plate (WilsonWolf, P/N 80192M) in 3 ml of Human T cell complete media containing 100 IU/ml IL-2, 10 ng/ml IL-7 and 25 ng/ml IL-15. On day 4 post transduction, the dynabeads were removed according to manufacturer's protocol. The TCR-T cells were seeded to Grex 6-well plate (WilsonWolf, P/N 80240M) at ˜10×10⁶ cells in 30 ml media per well in the presence of 100 IU/ml IL-2, 10 ng/ml IL-7 and 25 ng/ml IL-15. On day 7 post transduction, the TCR-Ts were harvested, frozen down and stored in liquid nitrogen vapor phase. TCR transduction efficiency were validated by dextramer binding.

### Flow Cytometry

The following antibodies were used for T cell phenotyping: CD3-FITC (Biolengend: 300440), CD8-BV605 (BD: 564116), CD4-PE (Biolegend: 317410). The following antibodies were used for dendric cell phenotyping: CD14-percpcy5.5 (Biolegend: 301824), CD11c-PE (Biolegend: 337206), CD1a-APC-cy7 (Biolegend: 300125), CD86-APC (BD: 555660). The following antibodies were used for B cell phenotyping: MHC class I (Biolegend: 311414), MHC class II (Biolengend: 361706), CD83-PE (BD 556855), CD86-APC (BD: 555660), CD20-FITC (BD: 556632). Dextramers-APC or -PE were purchased from Immudex (customized dextramers). 50 nM PKI dasatinib (Axon Medchem: 1392) was used to prevent TCR internalization. The TCR expressing T cells were incubated with 50 nM PKI dasatinib at 37° C. for 30 min and then followed by dextramer staining on ice for 30 min and cell surface marker staining at 4° C. for 15 min. The dead cell exclusion stain (Sytox blue, ThermoFisher/Invitrogen, S34857) was used. Flow cytometry data were analyzed using Flowjo.

### T2-Luc Killing Assay

Functionality and killing specificity of MAGE-A4 TCR-T was determined by T2-luc (T2 cell line expressing luciferase) killing assays. T2-luc cells were collected, washed and resuspended at 2×10⁶ cells/ml in T2-luc killing assay media (RPMI 1640-GlutaMAX, 1× Non-Essential Amino Acids Solution (Gibco, 11140-050), 10 mM HEPES (Gibco, 15630-080), 50 μM 2-ß-mercaptoethanol (Gibco, 21985-023), 1 mM sodium pyruvate (Gibco, 11360-070), 100 U/ml Penicillin-Streptomycin (Gibco, 15140-122), 5% heat-inactivated FBS (Gibco, 10082-147), and then seeded at 1 ml per well in 24-well plate. T2-Luc cells were pulsed with the indicated peptide concentrations for two to four hours at 37° C. T2-luc cells were then washed and resuspended at 1×10⁵ cells/ml and were seeded at 25 μl per well in 384-well plates (Corning, 3570). T2-Luc cells were incubated with 25 μl of TCR-T cells with the indicated dextramer+ TCR-T to T2-luc cells ratio for 48 hours. The luminescent signal was measured by addition of 30 μl of Bio-glo (Promega, G7940) followed by measurement of luminescent signals by using Biostack neo system (BioTek, Winooski, VT). Prior to the killing assays, all of different TCR-Ts were normalized to the same amount of MAGE-A4 dextramer+ cells (e.g. 10%) by adding mock (untransduced) T cells. Specific lysis (specific killing %) was calculated through normalization of TCR-T+T2/target peptide killing either by mock T cells+T2/target peptide killing or by TCR-T+T2/no peptide killing. Specific lysis formulas are described below.

### Formula for Specific Lysis (%)

### Peptide Titration (MAGE-A4 Peptides and Similar Peptides):

$$\{ 1 - ( TCRT + T 2 - luc / test peptide RLU ) / ( TCRT + T 2 - luc or C 1 R - luc / no peptide RLU ) \} \times 100$$

### Cancer Cell Line Killing:

$$\{ 1 - ( TCRT + cancer cell line RLU ) / ( cancer cell , \text{} cancer cell + mock , or cancer cell + RFP - IL 12 T cell controls ) \} \times 100$$

### Cancer Cell Killing Assay

Cytotoxicity of TCR-T cells against MAGE-A4 positive and negative cancer cell lines was determine by cancer cell killing assay. Cancer cells were collected, washed and resuspended at 1×10⁵ cells/ml in cancer cell killing assay media (RPMI 1640-GlutaMAX, 1× Non-Essential Amino Acids Solution (Gibco, 11140-050), 10 mM HEPES (Gibco, 15630-080), 50 μM 2-ß-mercaptoethanol (Gibco, 21985-023), 1 mM sodium pyruvate (Gibco, 11360-070), 100 U/ml Penicillin-Streptomycin (Gibco, 15140-122), 10% heat-inactivated FBS (Gibco, 10082-147). Cancer cells were then seeded at 25 μl per well in 384-well plates and incubated with 25 μl of TCR-T cells with the indicated dextramer+ TCR-T to T2-luc cells ratio for 48 hours. Following incubation, for adherent cancer cells, the suspension T cells were removed, and wells were washed with DPBS with Ca²⁺Mg²⁺ (Corning, 21-031-CM) using plate washer. The luminescent signal was measured by addition of 30 μl of Celltiter Glo (Promega, G7573). For suspension luciferase labeled cancer cells, the luminescent signal was measured by addition of 30 μl of Bio-glo (Promega, G7940). Biostack neo system was used for luminescence measurement. For suspension cancer cells without luciferase labeling, cancer cells were labeled by Celltrace far red (Invitrogen, Carlsbad, CA, USA). Cancer cells were resuspended in serum free RPMI media containing Celltrace far red (1:4000 dilution) at 1×10⁶ cells/ml and were incubated at 37° C. for 10 min. The reaction was stopped by adding 30 ml killing assay media and incubating at room temperature for 10 min. Live cancer cells were detected by flow cytometry. The dead cell exclusion stain (Sytox blue, ThermoFisher/Invitrogen, S34857) was used. Specific lysis (specific killing %) was calculated through normalization of TCR-T killing against a cancer cell line by mock T cell killing against a cancer cell line. Specific lysis formula is described above.

### Similar Peptide Screen

Functional specificity of MAGE-A4 TCR-T was determined using T2luc T-cell directed killing assays. Peptides including target and similar peptides were synthesized by JPT (Berlin, Germany) or AnaSpec (Fremont, CA). T2luc cells were incubated with reactive similar peptides, target specific peptide in assay media (RPMI 1640 supplemented with 5% heat inactivated FBS (MilliporeSigma), 1× GlutaMax (Gibco), 1× non-essential amino acids solution (Hyclone), 10 mM HEPES (Hyclone), 50 μM 2-ß-mercaptoethanol (Gibco), 1 mM sodium pyruvate (Gibco) at a final peptide concentration range of 1.0E-05M to 6.0E-16M (potency) or 1.0E-05M (single point) for 2 hours at 37° C./5% CO₂. Frozen MAGE-A4 TCR-T and mock cells were thawed, washed and rested in 50/50 RPMI/AIM-V/5% huAB serum, 1× GlutaMax, 25 mM HEPES, 100 u P/S (Gibco), 10 ug/mL gentamicin (Gibco) for 3 hrs prior to assay set-up. MAGE-A2 TCR-T cells were washed 3× in assay media and re-suspended at 2.5E06 cells/mL. Peptide loaded T2luc cells were added to white-clear bottom 384-well assay plates (Costar) at 2,000 cells/25 μL using Bravo liquid handling system (Agilent). MAGE-A4 TCR-T cells were prepared by diluting MAGE-A4 dextramer positive cells with mock T-cells to obtain a 10:1 target:effector ratio; 20,000 cells/25 μL (final 1:1 Dex+ Tcell:T2luc). T2luc pulsed cells and TCR-T cells were incubated for 48 hours at 37° C./5% CO2. T2luc cell viability was determined using Bio-GLo Luciferase Assay System (Promega, G7940) according to the manufacturer's recommendation. Luminescence was detected using En Vision Multilable Plate Reader (Perkin Elmer). Percent viability was calculated using the following formula: % Viability=(Sample raw RLU value/Average DMSO control RLU)×100. EC50 was determined using GraphPad Prism (non-linear regression curve fit analysis).

### Human Primary Normal Cell Culture

Sources of human primary normal cells and iPSC-derived cells are summarized in Table 5. Culture conditions for those cells are summarized in Table 5. Primary cells were thawed and cultured according to the supplier's instructions with the following exceptions: cardiomyocytes, astrocytes, GABA neurons, and RPTEC which were converted into RPMI 1640 culture medium just prior to the initiation of coculture. Prior optimization studies demonstrated a tolerability of RPMI 1640 and improvement in cell viability for these cell types. All cells were counted and assessed for viability prior to assay.

TABLE 5
Source of human normal primary and iPSC-derived cells

| Cells | Cell Type | Source | Donor | Catalog # |
|---|---|---|---|---|
| Bronchial | Primary | PromoCell | 424Z015.3 | C-12640 |
| Epithelial Cells | | | | |
| (hBEpC) | | | | |
| Renal Proximal | Primary | Lonza | 617045 | CC-2553 |
| Tubule Epithelial | | | | |
| Cells (RPTEC) | | | | |
| Tracheal | Primary | PromoCell | 446Z036.8 | C-12212 |
| Epithelial Cells | | | | |
| (hTEpC) | | | | |
| Keratinocytes | Primary | PromoCell | 425Z026.2 | C-12003 |
| Dermal | Primary | PromoCell | 435Z034.2 | C-12212 |
| Microvascular | | | | |
| Endothelial Cells | | | | |
| (HDMEC) | | | | |
| Hepatocytes | Primary | Lonza | HUM17299A, | HUCPG |
| | | | HUM173531 | |
| GABA Neurons | iPSC | Cellular | 01434 | R1013 |
| | | Dynamics | | |
| Astrocytes | iPSC | Cellular | 01434 | R1092 |
| | | Dynamics | | |
| Cardiomyocytes | iPSC | Cellular | 01434 | R1007 |
| | | Dynamics | | |
| B-CPAP | Thyroid carcinoma | DSMZ | N/A | N/A |
| | cell line | | | |
| | (MAGEB2⁺) | | | |

TABLE 6
Culture media and methods for human normal cells

| | | | | Plating Density |
|---|---|---|---|---|
| Cells | Assay Medium | Supplements | Specific Methods | (Cells/Well) |
| Bronchial | Airway | Required supplements | Plated cells directly | 20,000 |
| Epithelial Cells | Epithelial | contained in kit | into 96-well ViewPlates | |
| (hBEpC) | Cell Medium | (hydrocortisone | | |
| | | omitted) | | |
| Renal Proximal | RPMI with | 10% HI FBS, | Thawed and maintained | 20,000 |
| Tubule Epithelial | supplements | Pen/Strep | cells in REGM. | |
| Cells (RPTEC) | | | Plated cells directly | |
| | | | into 96-well ViewPlates | |
| Tracheal | Airway | Required supplements | Plated cells directly | 20,000 |
| Epithelial Cells | Epithelial | contained in kit | into 96-well ViewPlates | |
| (hTEpC) | Cell Medium | (hydrocortisone | | |
| | | omitted) | | |
| Keratinocytes | Keratinocyte | Required supplements | Plated cells directly | 20,000 |
| | Growth | contained in kit | into 96-well ViewPlates | |
| | Medium | (hydrocortisone | | |
| | | omitted) | | |
| Dermal | Endothelial | Required supplements | Plated cells directly | 20,000 |
| Microvascular | Cell Growth | contained in kit | into 96-well ViewPlates | |
| Endothelial Cells | Medium | (hydrocortisone | | |
| (HDMEC) | | omitted) | | |
| Hepatocytes | Hepatocyte | Required supplements | Thawed in Hepatocyte | 30,000 |
| | Maintenance | contained in kit | Thaw Medium; plated | |
| | Medium | (hydrocortisone | in William's Medium | |
| | | omitted) | E with Hepatocyte | |
| | | | Plating Supplements | |
| | | | into collagen-coated | |
| | | | 96-well ViewPlates; | |
| | | | after 24 hr incubation, | |
| | | | cells washed and | |
| | | | assayed in Hepatocyte | |
| | | | Maintenance Medium | |
| GABA Neurons | RPMI with | 10% HI FBS, | Plated directly in iCell | 20,000 |
| | supplements | Pen/Strep | Neural Base Medium | |
| | | | with Neural Supplement | |
| | | | A into 96-well PDL-coated | |
| | | | ViewPlates coated with | |
| | | | 3.33 ug/mL Laminin. | |
| | | | After 24 hr incubation, | |
| | | | cells were washed and | |
| | | | assayed in RPMI | |
| Astrocytes | RPMI with | 10% HI FBS, | Plated directly in DMEM | 20,000 |
| | supplements | Pen/Strep | with N-2 Supplement A | |
| | | | into 96-well ViewPlates. | |
| | | | After 24 hr incubation, | |
| | | | cells were washed and | |
| | | | assayed in RPMI | |
| Cardiomyocytes | RPMI with | 10% HI FBS, | Plated directly in iCell | 20,000 |
| | supplements | Pen/Strep | Cardiomyocyte Plating | |
| | | | media into 96-well | |
| | | | ViewPlates coated with | |
| | | | 0.1% gelatin. | |
| | | | After 24 hr incubation, | |
| | | | cells were washed with | |
| | | | iCell Cardiomyocyte | |
| | | | Maintenance Medium. | |
| | | | Media is replaced every | |
| | | | other day until spontaneous | |
| | | | beating is observed. | |
| | | | Cells were washed again | |
| | | | in Maintenance Media | |
| | | | and assayed in RPMI. | |
| B-CPAP | RPMI with | 10% HI FBS, | Plated cells directly | 20,000 |
| | supplements | Pen/Strep | into 96-well ViewPlates | |

Cytotoxicity Assays with Human Primary Normal Cells

Target cell cytotoxicity was assessed using a phase contrast/fluorescence kinetic imaging assay. Fluorescent caspase 3/7 cleavage was measured over time with an IncuCyte® live imaging device and overlaid onto phase contrast images that captured cell confluence. Prior to implementing the cytotoxicity assay, different plating densities and tolerability to various culture media were assessed to achieve suitable confluence without significant cell overlap in 96-well plates. Target cells (100 μl) were added at the densities listed in Table 3 to black 96-well ViewPlates containing 50 μl of MAGE-A4 TCR-T-IL12 cells, IL-12-RFP T cells, or mock T cells at a dextramer-normalized effector:target (E:T) ratio of 1:1, by taking into consideration the dextramer positivity of each TCR-T construct. CellEvent caspase 3/7 reagent (50 μl) was added according to the manufacturer's instructions (ThermoFIsher, C10423). Assay plates were placed in a 37° C., 5% CO2 incubator equipped with an IncuCyte® S3. Phase contrast and fluorescent images (5 fields) with the 10× objective were collected every 4 hours starting at 0 hour for 44 or 48 hours and analyzed for Caspase 3/7 total integrated intensity using IncuCyte® 2019B software. As T cells are generally smaller than target cells, a minimum area filter was set at 200 μm2 in fluorescent images to exclude signals from apoptotic T cells. In addition, since fluorescent signals in target cells were not homogeneous, target cells could be recognized as smaller splits and excluded by the area filter. Therefore, low edge detection sensitivity was also applied during analysis. After 44 or 48 hours, plates were removed from the incubator, and 50 μL of cell culture medium was removed from the wells for cytokine analysis.

### Alloreactivity Screen

Alloreactivity potential was assessed by co-culturing each of the 4 TCR-T cells with 34 BLCL lines (B lymphoblastoid cell lines) representing 39 HLA-A, 40 HLA-B, and 23 HLA-C alleles. BLCLs were purchased from Fred Hutchinson Cancer Research Institute (Fred Hutch; Seattle, WA) and Astarte Biologics (Cellero; Bothell, WA) as listed in Table 7. BLCLs were cultured in 15% FBS complete RPMI containing: RPMI-1640 with L-Glutamine, 15% (v/v) HI-FBS, and 1 mM Sodium Pyruvate.

U266B1 cells (ATCC; 10⁵ cells/ml in media) as a MAGE-A4+ HLA-A*02:01+ positive control cell line were pulsed with 50 μM MAGE-A4 peptide by incubation at 37° C. for 2 hours. TCR-T cells from donor 12665 were thawed by addition of media, centrifuged at 400×g for 5 min at 4° C., resuspended in 10 ml of media, and counted. TCR-T cells were co-cultured with either BLCLs or peptide-pulsed U266B1 cells in 200 μl volume. The dextramer-normalized effector:target ratios for the 4 TCR-T cells ranged from 3:1 to ˜8:1, depending upon the respective dextramer-positivity. All co-cultures were conducted in 96-well flat-bottom tissue culture plates at 37° C., 5% CO2 for 48 hours. Following incubation, the 96-well plates were centrifuged at 887×g for 1 min at 4° C. and the supernatant was collected into 96-well V-bottom plates for cytokine analysis. Cytokines and Granzyme B were evaluated by Luminex assay using a custom Milliplex Human Cytokine/Chemokine Kit (Millipore, SRP1885), including the analytes of IFNγ, granzyme B, TNFα, and IL-12p70, as per manufacturer instructions. Serial dilutions of analyte standards were run in replicates on each assay plate. The Luminex plate was read on a FlexMap 3D instrument (XMAP technologies). Data was exported by xPONENT Software, and analyzed directly by EMD Millipore's Milliplex Analyst software, generating standard curves using a 5-parameter logistic non-linear regression fitting curve. The limits of detection (Min and Max) were calculated by the Milliplex Analyst software as the result of the average of appropriate replicate standard curve values obtained from each assay plate and indicate the range within which an analyte can be interpolated from the standards. Samples were run at appropriate dilutions to ensure measurements of sample analyte levels were within assay standard curve limits. Cytokine and granzyme B levels are reported in pg/mL or as fold-differences over IL12-RFP T cells (controls) and graphed in GraphPad Prism software.

TABLE 6
BLCL lines for alloreactivity screen

| Cell Line Name | IHW Reference | Vendor |
|---|---|---|
| 1346-8357 | IHW01080 | Fred Hutch |
| 1347-8440 | IHW01103 | Fred Hutch |
| 1347-8442 | IHW01105 | Fred Hutch |
| 1416-1189 | IHW01176 | Fred Hutch |
| 1416-1337 | IHW01185 | Fred Hutch |
| FH19 | IHW09400 | Fred Hutch |
| FH31 | IHW09413 | Fred Hutch |
| FH39 | IHW09427 | Fred Hutch |
| FH46 | IHW09434 | Fred Hutch |
| FH70EY | IHW09458 | Fred Hutch |
| LCK | IHW09367 | Fred Hutch |
| TEM | IHW09057 | Fred Hutch |
| 165 | — | Astarte Biologics |
| FH18 | IHW09398 | Fred Hutch |
| FH21 | IHW09403 | Fred Hutch |
| FH25 | IHW09407 | Fred Hutch |
| FH3 | IHW09375 | Fred Hutch |
| FH36 | IHW09423 | Fred Hutch |
| FH43 | IHW09431 | Fred Hutch |
| FH53 | IHW09441 | Fred Hutch |
| FH6 | IHW09380 | Fred Hutch |
| FH9 | IHW09383 | Fred Hutch |
| ISH4 | IHW09371 | Fred Hutch |
| KT14 | IHW09103 | Fred Hutch |
| MYE 2003 | IHW09419 | Fred Hutch |
| MYE 2004 | IHW09420 | Fred Hutch |
| MYE 2006 | IHW09422 | Fred Hutch |
| SCL-116A | IHW09465 | Fred Hutch |
| T7526 | IHW09076 | Fred Hutch |
| TER-259 | IHW09401 | Fred Hutch |
| TUBO | IHW09045 | Fred Hutch |
| RSH | IHW09021 | Fred Hutch |
| WUZH1 | IHW09459 | Fred Hutch |

1. An expression vector comprising a nucleic acid sequence encoding a T-cell receptor (TCR) alpha chain and a TCR beta chain, wherein the TCR alpha chain and TCR beta chain are selected from the group consisting of:
a. a TCR alpha chain comprising a CDR1, 2, and 3 sequence comprising an amino acid sequence set forth in SEQ ID NO:3, 5, and 7, respectively, and a TCR beta chain comprising a CDR1, 2, and 3 sequence comprising an amino acid sequence set forth in SEQ ID NO: 4, 6, and 8, respectively;
b. a TCR alpha chain comprising a CDR1, 2, and 3 sequence comprising an amino acid sequence set forth in SEQ ID NO:13, 15, and 17, respectively, and a TCR beta chain comprising a CDR1, 2, and 3 sequence comprising an amino acid sequence set forth in SEQ ID NO: 14, 16, and 18, respectively;
c. a TCR alpha chain comprising a CDR1, 2, and 3 sequence comprising an amino acid sequence set forth in SEQ ID NO:23, 25, and 27, respectively, and a TCR beta chain comprising a CDR1, 2, and 3 sequence comprising an amino acid sequence set forth in SEQ ID NO: 24, 26, and 28, respectively;
d. a TCR alpha chain comprising a CDR1, 2, and 3 sequence comprising an amino acid sequence set forth in SEQ ID NO:33, 35, and 37, respectively, and a TCR beta chain comprising a CDR1, 2, and 3 sequence comprising an amino acid sequence set forth in SEQ ID NO: 34, 36, and 38, respectively;
e. a TCR alpha chain comprising a CDR1, 2, and 3 sequence comprising an amino acid sequence set forth in SEQ ID NO:43, 45, and 47, respectively, and a TCR beta chain comprising a CDR1, 2, and 3 sequence comprising an amino acid sequence set forth in SEQ ID NO: 44, 46, and 48, respectively;
f. a TCR alpha chain comprising a CDR1, 2, and 3 sequence comprising an amino acid sequence set forth in SEQ ID NO:53, 55, and 57, respectively, and a TCR beta chain comprising a CDR1, 2, and 3 sequence comprising an amino acid sequence set forth in SEQ ID NO: 54, 56, and 58, respectively;
g. a TCR alpha chain comprising a CDR1, 2, and 3 sequence comprising an amino acid sequence set forth in SEQ ID NO:63, 65, and 67, respectively, and a TCR beta chain comprising a CDR1, 2, and 3 sequence comprising an amino acid sequence set forth in SEQ ID NO: 64, 66, and 68, respectively; and
h. a TCR alpha chain comprising a CDR1, 2, and 3 sequence comprising an amino acid sequence set forth in SEQ ID NO:73, 75, and 77, respectively, and a TCR beta chain comprising a CDR1, 2, and 3 sequence comprising an amino acid sequence set forth in SEQ ID NO: 74, 76, and 78, respectively.

2. The expression vector of claim 1, further comprising a nucleic acid encoding interleukin-12 (IL-12) or a functional variant thereof.

3. The expression vector of claim 1 or claim 2, wherein the expression vector is a viral vector.

4. The expression vector of claim 3, wherein the viral vector is a retroviral vector.

5. The expression vector of claim 4, wherein the retroviral vector is a lentiviral vector.

6. The expression vector of any of claims 1-5, wherein the TCR alpha chain and TCR beta chain comprises an amino acid sequence selected from the group consisting of:
a. an amino acid sequence set forth in SEQ ID NO:9 or 10 and the TCR beta chain comprises an amino acid sequence set forth in SEQ ID NO:11 or 12;
b. an amino acid sequence set forth in SEQ ID NO: 19 or 20 and the TCR beta chain comprises an amino acid sequence set forth in SEQ ID NO:21 or 22;
c. an amino acid sequence set forth in SEQ ID NO:29 or 30 and the TCR beta chain comprises an amino acid sequence set forth in SEQ ID NO:31 or 32;
d. an amino acid sequence set forth in SEQ ID NO:39 or 40 and the TCR beta chain comprises an amino acid sequence set forth in SEQ ID NO:41 or 42;
e. an amino acid sequence set forth in SEQ ID NO:49 or 50 and the TCR beta chain comprises an amino acid sequence set forth in SEQ ID NO:51 or 52;
f. an amino acid sequence set forth in SEQ ID NO:59 or 60 and the TCR beta chain comprises an amino acid sequence set forth in SEQ ID NO:61 or 62;
g. an amino acid sequence set forth in SEQ ID NO:69 or 70 and the TCR beta chain comprises an amino acid sequence set forth in SEQ ID NO:71 or 72; and
h. an amino acid sequence set forth in SEQ ID NO:79 or 80 and the TCR beta chain comprises an amino acid sequence set forth in SEQ ID NO:81 or 82.

7. A cell expressing a recombinant T-cell receptor (TCR), said TCR comprising a TCR alpha chain and a TCR beta chain, wherein the TCR alpha chain and TCR beta chain are selected from the group consisting of:
a. a TCR alpha chain comprising a CDR1, 2, and 3 sequence comprising an amino acid sequence set forth in SEQ ID NO:3, 5, and 7, respectively, and a TCR beta chain comprising a CDR1, 2, and 3 sequence comprising an amino acid sequence set forth in SEQ ID NO: 4, 6, and 8, respectively;
b. a TCR alpha chain comprising a CDR1, 2, and 3 sequence comprising an amino acid sequence set forth in SEQ ID NO:13, 15, and 17, respectively, and a TCR beta chain comprising a CDR1, 2, and 3 sequence comprising an amino acid sequence set forth in SEQ ID NO: 14, 16, and 18, respectively;
c. a TCR alpha chain comprising a CDR1, 2, and 3 sequence comprising an amino acid sequence set forth in SEQ ID NO:23, 25, and 27, respectively, and a TCR beta chain comprising a CDR1, 2, and 3 sequence comprising an amino acid sequence set forth in SEQ ID NO: 24, 26, and 28, respectively;
d. a TCR alpha chain comprising a CDR1, 2, and 3 sequence comprising an amino acid sequence set forth in SEQ ID NO:33, 35, and 37, respectively, and a TCR beta chain comprising a CDR1, 2, and 3 sequence comprising an amino acid sequence set forth in SEQ ID NO: 34, 36, and 38, respectively;
e. a TCR alpha chain comprising a CDR1, 2, and 3 sequence comprising an amino acid sequence set forth in SEQ ID NO:43, 45, and 47, respectively, and a TCR beta chain comprising a CDR1, 2, and 3 sequence comprising an amino acid sequence set forth in SEQ ID NO: 44, 46, and 48, respectively;
f. a TCR alpha chain comprising a CDR1, 2, and 3 sequence comprising an amino acid sequence set forth in SEQ ID NO:53, 55, and 57, respectively, and a TCR beta chain comprising a CDR1, 2, and 3 sequence comprising an amino acid sequence set forth in SEQ ID NO: 54, 56, and 58, respectively;
g. a TCR alpha chain comprising a CDR1, 2, and 3 sequence comprising an amino acid sequence set forth in SEQ ID NO:63, 65, and 67, respectively, and a TCR beta chain comprising a CDR1, 2, and 3 sequence comprising an amino acid sequence set forth in SEQ ID NO: 64, 66, and 68, respectively; and
h. a TCR alpha chain comprising a CDR1, 2, and 3 sequence comprising an amino acid sequence set forth in SEQ ID NO:73, 75, and 77, respectively, and a TCR beta chain comprising a CDR1, 2, and 3 sequence comprising an amino acid sequence set forth in SEQ ID NO: 74, 76, and 78, respectively.

8. The cell of claim 7, wherein the TCR alpha chain and TCR beta chain comprises an amino acid sequence selected from the group consisting of:
a. an amino acid sequence set forth in SEQ ID NO:9 or 10 and the TCR beta chain comprises an amino acid sequence set forth in SEQ ID NO:11 or 12;
b. an amino acid sequence set forth in SEQ ID NO: 19 or 20 and the TCR beta chain comprises an amino acid sequence set forth in SEQ ID NO:21 or 22;
c. an amino acid sequence set forth in SEQ ID NO:29 or 30 and the TCR beta chain comprises an amino acid sequence set forth in SEQ ID NO:31 or 32;
d. an amino acid sequence set forth in SEQ ID NO:39 or 40 and the TCR beta chain comprises an amino acid sequence set forth in SEQ ID NO:41 or 42;
e. an amino acid sequence set forth in SEQ ID NO:49 or 50 and the TCR beta chain comprises an amino acid sequence set forth in SEQ ID NO:51 or 52;
f. an amino acid sequence set forth in SEQ ID NO:59 or 60 and the TCR beta chain comprises an amino acid sequence set forth in SEQ ID NO:61 or 62;
g. an amino acid sequence set forth in SEQ ID NO:69 or 70 and the TCR beta chain comprises an amino acid sequence set forth in SEQ ID NO:71 or 72; and
h. an amino acid sequence set forth in SEQ ID NO: 79 or 80 and the TCR beta chain comprises an amino acid sequence set forth in SEQ ID NO:81 or 82.

9. The cell of claim 7 or 8, wherein the cell further expresses a recombinant IL-12 or functional variant thereof.

10. A cell comprising an expression vector of any of claims 1-6.

11. The cell of any of claims 7-10, wherein the cell is a T cell.

12. The cell of claim 11, wherein the TCR binds the peptide of SEQ ID NO:1 or SEQ ID NO:2 in the context of HLA-A*0201 and said binding leads to activation of IFNγ, TNFα, IL-12, or granzyme B production by said cell.

13. A pharmaceutical composition comprising a therapeutically effective amount of a cell of any of claims 7-12.

14. A method of making a cell of any of claims 7-12 or a pharmaceutical composition of claim 13, comprising introducing into a cell an expression vector comprising a nucleic acid sequence encoding a TCR alpha chain and a TCR beta chain, wherein the TCR alpha chain and TCR beta chain are selected from the group consisting of:
a. a TCR alpha chain comprising a CDR1, 2, and 3 sequence comprising an amino acid sequence set forth in SEQ ID NO:3, 5, and 7, respectively, and a TCR beta chain comprising a CDR1, 2, and 3 sequence comprising an amino acid sequence set forth in SEQ ID NO: 4, 6, and 8, respectively;
b. a TCR alpha chain comprising a CDR1, 2, and 3 sequence comprising an amino acid sequence set forth in SEQ ID NO:13, 15, and 17, respectively, and a TCR beta chain comprising a CDR1, 2, and 3 sequence comprising an amino acid sequence set forth in SEQ ID NO: 14, 16, and 18, respectively;
c. a TCR alpha chain comprising a CDR1, 2, and 3 sequence comprising an amino acid sequence set forth in SEQ ID NO:23, 25, and 27, respectively, and a TCR beta chain comprising a CDR1, 2, and 3 sequence comprising an amino acid sequence set forth in SEQ ID NO: 24, 26, and 28, respectively;
d. a TCR alpha chain comprising a CDR1, 2, and 3 sequence comprising an amino acid sequence set forth in SEQ ID NO:33, 35, and 37, respectively, and a TCR beta chain comprising a CDR1, 2, and 3 sequence comprising an amino acid sequence set forth in SEQ ID NO: 34, 36, and 38, respectively;
e. a TCR alpha chain comprising a CDR1, 2, and 3 sequence comprising an amino acid sequence set forth in SEQ ID NO:43, 45, and 47, respectively, and a TCR beta chain comprising a CDR1, 2, and 3 sequence comprising an amino acid sequence set forth in SEQ ID NO: 44, 46, and 48, respectively;
f. a TCR alpha chain comprising a CDR1, 2, and 3 sequence comprising an amino acid sequence set forth in SEQ ID NO:53, 55, and 57, respectively, and a TCR beta chain comprising a CDR1, 2, and 3 sequence comprising an amino acid sequence set forth in SEQ ID NO: 54, 56, and 58, respectively;
g. a TCR alpha chain comprising a CDR1, 2, and 3 sequence comprising an amino acid sequence set forth in SEQ ID NO:63, 65, and 67, respectively, and a TCR beta chain comprising a CDR1, 2, and 3 sequence comprising an amino acid sequence set forth in SEQ ID NO: 64, 66, and 68, respectively; and
h. a TCR alpha chain comprising a CDR1, 2, and 3 sequence comprising an amino acid sequence set forth in SEQ ID NO:73, 75, and 77, respectively, and a TCR beta chain comprising a CDR1, 2, and 3 sequence comprising an amino acid sequence set forth in SEQ ID NO: 74, 76, and 78, respectively.

15. The method of claim 14, wherein the TCR alpha chain and TCR beta chain are selected from the group consisting of:
a. an amino acid sequence set forth in SEQ ID NO:9 or 10 and the TCR beta chain comprises an amino acid sequence set forth in SEQ ID NO:11 or 12;
b. an amino acid sequence set forth in SEQ ID NO: 19 or 20 and the TCR beta chain comprises an amino acid sequence set forth in SEQ ID NO:21 or 22;
c. an amino acid sequence set forth in SEQ ID NO:29 or 30 and the TCR beta chain comprises an amino acid sequence set forth in SEQ ID NO:31 or 32;
d. an amino acid sequence set forth in SEQ ID NO:39 or 40 and the TCR beta chain comprises an amino acid sequence set forth in SEQ ID NO:41 or 42;
e. an amino acid sequence set forth in SEQ ID NO:49 or 50 and the TCR beta chain comprises an amino acid sequence set forth in SEQ ID NO:51 or 52;
f. an amino acid sequence set forth in SEQ ID NO:59 or 60 and the TCR beta chain comprises an amino acid sequence set forth in SEQ ID NO:61 or 62;
g. an amino acid sequence set forth in SEQ ID NO:69 or 70 and the TCR beta chain comprises an amino acid sequence set forth in SEQ ID NO:71 or 72; and
h. an amino acid sequence set forth in SEQ ID NO: 79 or 80 and the TCR beta chain comprises an amino acid sequence set forth in SEQ ID NO:81 or 82.

16. The method of claim 14 or 15, wherein the expression vector further comprises a nucleic acid sequence encoding IL-12 or a functional variant thereof.

17. The method of any of claims 14-16, wherein the cell is a T cell.

18. The method of claim 17, wherein the T cell is a primary T cell.

19. The method of 18, wherein the primary T cell is isolated from a cancer patient.

20. A method of treating a MAGE-A4 or MAGE-A8 expressing cancer, said method comprising administering to a cancer patient a therapeutically effective amount of a cell of any of claims 7-12, of a pharmaceutical composition of claim 13, or of a cell made by the method of any of claims 14-19.

21. The method of claim 20, wherein the patient is tested prior to administration to determine the presence of a cancer expressing MAGE-A4 or MAGE-A8.

22. The method of claim 21, wherein a nucleic acid encoding MAGE-A4 or MAGE-A8 is detected.

23. The method of claim 21, wherein MAGE-A4 or MAGE-A8 protein or a MAGE-A4-derived or MAGE-A8-derived peptide is detected.

24. The method of any of claims 20-23, wherein the patient is identified to carry the HLA-A*0201 allele.
